(19) United States
(12) Patent Application Publication
(10) Pub. No.: US 2026/0000684 A1
(43) Pub. Date: Jan. 1, 2026

(54) CANNABINOID COMPOSITIONS AGAINST CANCER, THEIR IDENTIFICATION AND PERSONALIZATION OF CANNABIS-BASED CANCER THERAPY

(71) Applicant: UNIVERZA V MARIBORU, MARIBOR (SI)

(72) Inventors: Jan ROŽANC, MARIBOR (SI); Marko MILOJEVIC, MARIBOR (SI); Marko JUKIC, MARIBOR (SI); Urban BREN, MARIBOR (SI); Uroš MAVER, MARIBOR (SI)

(21) Appl. No.: 18/879,851

(22) PCT Filed: Jun. 30, 2023
(86) PCT No.: PCT/EP2023/068051

§ 371 Date: Dec. 30, 2024

(30) Foreign Application Priority Data
Jun. 30, 2022 (LU) LU502426

(51) Int. Cl. A61K 31/00; A61K 36/185; A61P 35/00; G01N 33/574; G01N 33/94; G16C 20/30; G16H 10/40

(52) U.S. Cl. CPC A61K 31/658; A61K 36/3482; A61P 35/00; G01N 33/57484; G01N 33/948; G16C 20/30; G16H 10/40

(57) ABSTRACT

In a first aspect, the invention pertains to a method for identifying a therapeutic composition against cancer comprising at least one cannabinoid. It is based on a holistic approach that relies on determining its therapeutic efficacy of a composition comprising at least one cannabinoid and by performing a computational analysis on the resulting data. In additional aspects, the invention pertains to therapeutic compositions comprising at least one cannabinoid and their use for the treatment of a condition or disease. Furthermore, the invention pertains to an in-vitro method for the personalization of cannabis-based therapy. Said method relies on providing a patient sample and performing a multiomic analysis on said patient sample. After predicting the patient's response to a therapeutic composition comprising at least one cannabinoid, a therapeutic composition comprising at least one cannabinoid is selected for treatment of the patient.

### FIELD OF THE INVENTION

In a first aspect, the invention pertains to a method for identifying a therapeutic composition against cancer comprising at least one cannabinoid. It is based on a holistic approach that relies on determining the therapeutic efficacy of a composition comprising at least one cannabinoid and by performing a computational analysis on the resulting data. In additional aspects, the invention pertains to therapeutic compositions comprising at least one cannabinoid and their use for the treatment of a condition or disease. Furthermore, the invention pertains to an in-vitro method for the personalization of cannabis-based therapy. Said method relies on providing a patient sample and performing a multiomic analysis on said patient sample. After predicting the patient's response to a therapeutic composition comprising at least one cannabinoid, a therapeutic composition comprising at least one cannabinoid is selected for treatment of the patient.

### DESCRIPTION

Cannabis sativa is a chemically complex species based on its numerous reported natural secondary metabolite constituents. The Cannabis sativa plant contains over 144 cannabinoids and hundreds of other compounds with therapeutic potential. The cannabinoids have conventionally been classified into 11 general types according to their chemical structures. The biological effects and interplay of these compounds are far from fully understood, although the plant's therapeutic effects are beyond doubt. In addition to (−)-Δ9-trans-tetrahydrocannabinol (Δ9-THC), one of the principal psychotropic ingredients in the plant, and cannabidiol (CBD), which have both already reached clinical application as sole pharmacotherapeutic agents or in combination, many other phytocannabinoids found in Cannabis are less studied but still presumed to have pharmacological properties either on their own or adding to synergistic effects of cannabis extracts.

The human endocannabinoid system (ECS) is a complex, evolutionarily conserved homeostatic signaling network that acts as a broad-spectrum modulator with numerous interactions that affect the physiology and pathology of the human body. One of its main functions is the regulation of cell, tissue, organ, and organism homeostasis. The ECS comprises endogenous ligands (endocannabinoids [eCBs], e.g. anandamide [AEA] and 2-arachidonoylglycerol [2-AG]), eCB-responsive receptors (e.g. CB1 and CB2 cannabinoid receptors), and a complex enzyme (e.g., fatty acid amide hydrolase [FAAH] and monoacylglycerol lipase [MAG lipase]) and transporter apparatus (e.g., heat shock proteins [HSPs] and fatty acid-binding proteins [FABPs]. The ECS and endocannabinoids play a role in various physiological and pathological processes, such as regulating excitatory and inhibitory synaptic transmission in the central nervous system, food intake, nociceptive signaling, analgesia, immunomodulation, inflammation, and cancer cell signaling. It has therefore emerged as an important target for the treatment of many diverse diseases, including cancer.

Besides eCBs and related endogenous mediators, the Cannabinaceae-derived phytocannabinoids (pCBs) represent another group of cannabinoids that can modulate ESC. Depending on their concentration, eCBs and pCBs can activate/antagonize/inhibit a remarkably wide variety of cellular targets, including several metabotropic (e.g., CB1 or CB2), ionotropic (certain transient receptor potential [TRP] ion channels), and nuclear (peroxisome proliferator-activated receptors (PPARs)) receptors, various enzymes, and transporters. Importantly, each ligand can be characterized by a unique molecular fingerprint, and in some cases, they can even exert opposing biological actions on the same target molecule.

The biological role of the ECS in cancer pathophysiology is not completely understood. Although there are some exceptions, most studies suggest that CB receptors and their endogenous ligands are upregulated in tumor tissue and that the overexpression of ECS components (i.e., receptors, ligands, and enzymes) correlates with tumor aggressiveness (1, 2), suggesting that the endocannabinoid system may be over-activated in cancer and are hence pro-tumorigenic (2).

In support of this, in mouse models of cancer, genetic ablation of CB1 and CB2 receptors reduced UV light-induced skin carcinogenesis (3), and CB2 receptor over-expression enhanced the predisposition to leukemia after leukemia virus infection (4). Conversely, and in line with evidence supporting that pharmacological activation of cannabinoid receptors reduces tumor growth, the up-regulation of endocannabinoid-degrading enzymes has been observed in aggressive human tumors and cancer cell lines, indicating that endocannabinoid signaling can also have a tumor-suppressive role.

Many forms of cannabinoids, including phytocannabinoids (e.g., THC, CBD, CBN), endocannabinoids (e.g., AEA, 2-AG), and synthetic cannabinoids (e.g. JWH-133, WIN-55,212-2), have shown the ability to inhibit proliferation, metastasis, and angiogenesis in a variety of cancer models. These findings strongly support that, aside from the role played by the endogenous cannabinoid system in cancer, pharmacological stimulation of CB receptors is, in most cases anti-tumorigenic. Cannabinoid-induced ER stress and autophagy were shown to be involved in several cancer types and play a role upstream of apoptosis in the process of cannabinoid receptor agonist-induced cell death.

In vivo experiments have shown that cannabinoids impair tumour angiogenesis by blocking the activation of the vascular endothelial growth factor (VEGF) pathway, an inducer of angiogenesis. This was demonstrated in skin carcinomas, gliomas and thyroid carcinomas with CB1 and/or CB2 receptor agonists downregulating the expression of its main ligand (VEGF) and the active forms of its main receptors (VEGFR1 and VEGFR2) (5). Likewise, CB1 and/or CB2 receptor agonists reduce the formation of distant tumor masses in animal models of both induced and spontaneous metastasis and inhibit adhesion, migration and invasiveness of glioma (6), breast (7), lung and cervical (8) cancer cells in culture.

Additionally, in a previous study, the inventors were able to show that Cannabis extracts can decrease viability in a colorectal adenocarcinoma cell line while not affecting the viability of untransformed intestinal epithelium cells at the same concentrations. Such difference in response to cannabinoid receptor stimulation seems to be coupled to the activation of different signaling mechanisms in transformed and non-transformed cells. However, the precise molecular reasons for this different behavior remain an important open question in the field. (9)

Like with any other cancer therapies, identifying resistance mechanisms to antitumor drug actions due to tumor heterogeneity has been a pressing issue. A study on a collection of glioma tumors identified that, although the apoptotic effect of cannabinoids relied on the stimulation of cannabinoid receptors and the activation of the autophagy pathway, the differences in the sensitivity correlated with expression of a particular set of genes (MDK and ALK), which were overexpressed in resistant cells, rather than with the presence of different expression levels of cannabinoid receptors (10). The study suggested that co-targeting the MDK-ALK pathway could significantly improve the glioma treatment outcome and could also point towards the need to identify resistance mechanisms in other cancer types. Such combinational anticancer therapies have several, currently at least theoretical, advantages over single-agent-based strategies, as they allow the simultaneous targeting of tumor growth, progression, and spreading at signaling and/or orchestration modulation levels.

Overall, besides suggesting that pCBs work through different pathways and receptors, which vary in different cancer cell populations, recent studies have show the advantage of using mixtures of Cannabis compounds over specific isolates. These findings point towards a synergistic effect of various Cannabis compounds named “the entourage effect” and indicate that the effect of a such a mixture on a specific cancer cell line relies on the mixtures' composition.

Malignant melanoma is the most lethal form of skin cancer, accounting for 90% of skin cancer-related deaths, and its global incidence continues to increase (11). It is a cancer, in which a multilevel orchestration of different signalling pathways seems to be present and is also the reason for a low efficiency of treatment approaches.

Identification of driver mutations and the development of targeted therapies to protooncogene B-Raf (BRAF)/mitogen-activated protein kinase (MEK) have revolutionized melanoma therapy, although clinical resistance is inevitable. Only recently, the emergence of immunotherapies that can promote tumor T-cell responses (e.g., programmed cell death protein 1 [PD-1] inhibitors), has lead to an increase in the overall survival rates from less than 10% to 40-50%. However, not all patients are responding to such a therapy due to, until now, not completely understood reasons. While great strides in melanoma therapy have generated some enthusiasm, many challenges with its treatment remain, listing it still among the most aggressive and therapy-resistant human cancers. Besides carrying one of the highest mutational burdens of all cancers, the melanoma tumor heterogeneity presents a pressing issue for all therapy types.

Several studies demonstrated that appropriate modulation of the complex cannabinoid signalling might exert anti-tumor activity in the case of melanoma. Human melanomas and melanoma cell lines express both CB1 and CB2 cannabinoid receptors, or they can even be found upregulated as compared to nevi or normal melanocytes.

Activation of eCB receptors has been shown to decrease growth, proliferation, angiogenesis, and metastasis, and increase apoptosis of melanomas in vitro and in vivo. Armstrong et al. showed that treatment with THC in human A375, SK-MEL-28, and CHL-1 melanoma cell lines resulted in the activation of autophagy, loss of cell viability, and activation of apoptosis, pointing to activation of non-canonical autophagy-mediated apoptosis of melanoma cells. They furthermore showed that the combination of THC and CBD in mice bearing BRAF wild-type melanoma xenografts has proven to be even more efficient as it substantially inhibited melanoma viability, proliferation, and tumor growth paralleled by an increase in autophagy and apoptosis compared with standard single-agent temozolomide (12).

In human melanoma cell lines expressing CB1 and CB2 cannabinoid receptors, the activation of those receptors led to anti-proliferative action due, at least in part, to cell cycle arrest at the G1-S transition via inhibition of the pro-survival protein AKT and hypophosphorylation of the pRB retinoblastoma protein tumor suppressor (13). Furthermore, the treatment of the CB2 and CB1 expressing melanoma cell lines (COLO38 and OCM-1) with synthetic cannabinoid receptor agonist WIN55,212-2 have been shown to reduce viability in a CB1/CB2-independent manner and induced activation of caspases and phosphorylation of the ERK1/2 MAPK cascade.

Zhou et al. showed that cannabinoids can inhibit melanogenesis and tyrosinase activity in α-MSH-stimulated B16 mouse melanoma cells in a PPARα-independent manner. Its effects were found to be coupled to the activation of p38 and ERK1/2 MAPK, as well as of Akt signalling cascades, and inhibition of the CREB pathway (14), pointing to a complex regulatory cannabinoid signalling role in melanocytes and melanoma. Such signalling pathways have been shown to regulate several other important cellular processes implicated in cell growth, proliferation, invasiveness, angiogenesis, and others and interact with parallel signalling pathways, indicating a need for a broader examination of signalling events in Cannabis mediated anti-tumor effects.

Colorectal cancer is considered the third most deadly and the fourth most commonly detected cancer in the world and has no effective curative options (15). While immunotherapy is a promising treatment option for this type of cancer, cancer cells may become resistant to immunotherapy and escape immune surveillance by obtaining genetic alterations. Targeting the ECS is a promising new strategy to in the treatment of colorectal cancer. The ECS actively regulates gut homeostasis, with its components being highly expressed in the intestinal tissue. For example, CB1 and CB2 receptors are expressed in healthy colon epithelium, submucosal myenteric plexus, smooth muscles, and plasma cells in the lamina propria; CB2 receptors are also present on the intestinal macrophages. TRPV1 receptors are expressed on colonic nerve fibers. The GPR55 receptor is present in the mucosa and the muscle layer of the colon. The endocannabinoids, 2-AG and AEA are also present in healthy colonic tissue. Components of ECS are significantly dysregulated in colorectal cancer, suggesting a potential impact of cannabinoids in the treatment of this disease.

Despite numerous studies conducted during the last decade, the obtained data regarding the endocannabinoid system's exact role in cancer development is still very inconsistent and sometimes even contradictory. Different cannabis compositions, such as Cannabis extracts have different effects on the survival of cancer cells. While some cancer cell types clearly respond to treatment with cannabis compositions, there are currently no known biomarkers that could predict treatment outcomes.

One of the most pressing questions related to the potential use of phytocannabinoids in cannabinoid compositions in cancer treatment is how these can modulate anticancer activity by orchestrating the interplay of the endocannabinoid system with the oncogenic signaling. Several signaling pathways that regulate proliferation (mitogen-activated protein kinase [MAPK]), autophagy (phosphatidylinositol 3-kinase [PI3K]/RAC-alpha serine/threonine-protein kinase [AKT]/mammalian target of rapamycin [mTOR]), apoptosis (prostaglandin-endoperoxide synthase 2 [COX2]/prostaglandin D2 synthase [PGD2]/peroxisome proliferator-activated receptor [PPAR]), angiogenesis (focal adhesion kinase [FAK]/proto-oncogene tyrosine-protein kinase Src [SRC]/vascular endothelial growth factor [VEGF]), cell cycle (G1/S-Specific Cyclin-E1 [CycE]/cyclin-dependent kinase 2 [CDK2]), invasion (SRY-Box transcription factor 2 [SOX2]) and metastasis (intercellular adhesion molecule 1 [ICAM-1]) have been shown to mediate or are involved in anticancerogenic effects of cannabinoids.

Further studies analyzing the activation of the precise signalling mechanisms involved in regulating cannabinoid-induced cell death or cell proliferation upon pharmacological manipulation of the endocannabinoid system are needed to clarify which are the contextual determinants for this system to act as either a guardian or an inducer of tumorigenesis or tumor progression. It is therefore necessary to understand these events on a system-based level to suggest optimal cannabinoid formulations for specific cancer types and identify new targets and corresponding biomarkers of response. It is of utmost importance to study these effects in systematic studies to shed more light on the exact biological pathways behind them and hence to understand the pharmacodynamics behind this activity. New therapeutic approaches that can cope with the above-described treatment resistance and heterogeneity are highly sought for, especially in melanoma therapy, and a personalized cannabis-based approach could present a promising new treatment option.

Thus, it is an object of the invention, to provide a method to systematically address these questions. The method of the inventors is based on describing the dynamic interactions of cannabinoid compounds with the complex system of the human body, taking into account all available data at different levels ranging from holistic clinical data to molecular studies of the interactions of cannabinoids with their respective targets. The method comprises comprehensive assaying of a biological samples, preferably obtained from a patient, to determine the abundance of a panel of biomarkers comprising genetic, transcriptomic, and protein markers. Using this holistic approach, the invention pertains to a method that may be used to identify and/or optimize therapeutic compositions comprising at least one cannabinoid, in particular which are suitable for use in the treatment of cancer. Furthermore, the method involves the input of the determined biomarker presence and abundance into a computational model aiming at quantifying and/or predicting with a high probability the individual's response to specific treatment. By determining and analyzing signalling pathway activity involved in oncogenic and cannabinoid signalling the inventors disclose a method for selecting personalized cannabis-based anti-cancer therapy.

### BRIEF DESCRIPTION OF THE INVENTION

Generally, and by way of brief description, the main aspects of the present invention can be described as follows:

In a first aspect, the invention pertains to a method for identifying a therapeutic composition against cancer comprising at least one cannabinoid, wherein the method comprises the steps:
(i) Determining a therapeutic efficacy of the composition comprising at least one cannabinoid with a predetermined chemical profile,
(ii) Performing a computational analysis on the therapeutic efficacy of the composition comprising at least one cannabinoid with the predetermined chemical profile.

In a second aspect, the invention pertains to a therapeutic composition against cancer comprising at least one cannabinoid.

In a third aspect, the invention pertains to a therapeutic composition against cancer comprising at least one cannabinoid, for use in the treatment of a condition or disease, preferably wherein the therapeutic composition against cancer comprising at least one cannabinoid is the therapeutic composition against cancer comprising at least one cannabinoid according to any other aspect of the invention.

In a fourth aspect, the invention pertains to an in vitro method for the personalization of cannabis-based cancer therapy, wherein the method comprises the steps:
(a) Providing a patient sample,
(b) Performing a multiomic analysis on said patient sample,
(c) Predicting the patient's response to a therapeutic composition comprising at least one cannabinoid, and
(d) Selecting a therapeutic composition comprising at least one cannabinoid for treatment of the patient based on the prediction of the patient's response.

In a fifth aspect, the invention pertains to a method for identifying and/or optimizing a therapeutic composition against cancer comprising at least one cannabinoid, wherein the method comprises the steps:
(i) Manufacturing a composition comprising at least one cannabinoid with a predetermined chemical profile,
(ii) Determining a therapeutic efficacy of the composition comprising at least one cannabinoid.
(iii) Performing a computational analysis on the data obtained in step (i) or (ii).

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for identifying a therapeutic composition against cancer comprising at least one cannabinoid, wherein the method comprises the steps:
(i) Determining a therapeutic efficacy of the composition comprising at least one cannabinoid with a predetermined chemical profile,
(ii) Performing a computational analysis on the therapeutic efficacy of the composition comprising at least one cannabinoid with the predetermined chemical profile.

In the context of the invention, “therapeutic composition” is meant to indicate that the composition comprising at least one cannabinoid exhibits a therapeutic effect. A therapeutic effect refers to a response of an individual or a cell, when being administered and/or exposed to the composition comprising at least one cannabinoid, which is regarded useful or favorable.

Preferably, the individual that is suffering from a disease such as cancer and/or the cell is a cancer cell. Examples of a therapeutic effect are cell death, changes in cell proliferation, changes in the gene expression such as the expression of biomarkers, expression of therapeutic targets, changes in metabolic pathways. “Against cancer” in this context indicates that the therapeutic composition is suitable for use in the treatment of cancer and/or a disease associated with cancer.

The “chemical profile” according to the invention refers to the relative and absolute abundance of chemical compounds in the composition comprising at least one cannabinoid. Preferably, it also includes solvent molecules. Alternatively, the “chemical profile” refers to a spectrum, preferably a MS spectrum or a spectroscopic spectrum, that can be used to uniquely identify a composition comprising at least one cannabinoid by its certain relative and/or absolute abundance of chemical compounds.

In preferred embodiments of the invention, the determining a therapeutic efficacy comprises comparing a biological marker of a sample that is not exposed to the composition comprising at least one cannabinoid to the biological marker of a sample that is exposed to the composition comprising at least one cannabinoid, wherein the increase and/or decrease of the biological marker is indicative of a therapeutic effect.

Preferably, the therapeutic efficacy is a therapeutic efficacy against cancer and/or cancer cells. In this context, the sample may be a solid tissue sample, a soft tissue sample, a bodily fluid and/or a cell. In preferred embodiments said sample is a patient sample. Preferably, the sample comprises a cell. The cell may be a healthy, diseased and/or a cancer cell. Preferably, the sample comprises a healthy cell. Preferably the sample comprises a cancer cell. 35

In preferred embodiments of the invention, the therapeutic effect is cancer cell death, impairment of tumor angiogenesis and/or inhibition of cancer cell proliferation, cancer cell adhesion, cancer cell migration, cell invasiveness and/or other harmful effects of cancer cells. In further preferred special embodiments of the invention, the therapeutic effect is cancer cell death, cancer cell viability, impairment of tumor angiogenesis and/or inhibition of cancer cell proliferation, cancer cell adhesion, cancer cell migration, cell invasiveness and/or other harmful effects of cancer cells.

In further preferred embodiments, the therapeutic effect is a higher rate of cancer cell death and/or inhibition of cancer cell proliferation, cancer cell adhesion and/or cancer cell migration compared to the rate of cell death and/or inhibition of cell proliferation, cell adhesion and/or cell migration of a non-cancer cell, preferably a healthy cell.

In preferred embodiments of the invention, the determining a therapeutic efficacy comprises conducting an assay that is suitable for the detection of a cancer cell function, preferably wherein the cancer cell function is cell proliferation and/or cell death. Preferably, cell proliferation is detected using an assay selected from the group of tetrazolium reduction assay, resazurin reduction assay, protease viability marker assay, and ATP assay. Preferably, cell proliferation is detected using an assay selected from the group of nucleoside-analogue incorporation assay, cell cycle-associated protein assay and cytoplasmic proliferation dye-based assay.

In preferred embodiments of the invention, the comparing a biological marker comprises conducting a multiomic analysis. A multiomics analysis, as used in the context of the invention describes a combinatory analysis method in which multiple “omes”, for example the genome, proteome, transcriptome, epigenome, metabolome and microbiome of a sample may be studied. In special preferred embodiments, the multiomic analysis comprises a spatial single cell profiling of the sample, a cell composition analysis, a cytokine expression profiling and/or a protein phosphorylation analysis. In special preferred embodiments, the multiomic analysis comprises a cell profiling of the sample, a cell composition analysis, a gene expression analysis, a neighborhood analysis, a cytokine expression profiling and/or a protein phosphorylation analysis.

In preferred embodiments of the invention, the multiomic analysis comprises spatial single cell profiling of the sample, preferably using a multiplexed immunohistochemistry method. In preferred embodiments of the invention, the multiplexed immunohistochemistry method comprises a detection, optionally a quantification, of a cannabinoid-related receptor. Preferably, the cannabinoid-related receptor is selected from at least one cannabinoid-related receptor of the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA), PPARβ (also called PPARD) and PPARγ (also called PPARG). In special preferred embodiments, the cannabinoid-related receptor is CBR1, CBR2, TRPV1, TRPV2, TRPV3, TRPV4, TRPA1, TRPVA1, TRPM8, GPR3, GPR6, GPR12, GPR18, GPR23, GPR35, GPR55, GPR84, GPR92, GPR119, PPARα, PPARβ and/or PPARγ. In special preferred embodiments, the cannabinoid-related receptor is selected from a receptor class of the group consisting of hydrolase, transferase, oxidoreductase, signaling protein, cell adhesion protein, transport protein, nuclear receptor, metalloprotease, ligase, matrix metalloprotease, flavoprotein, cell cycle related protein, lyase, isomerase, protein involved in immunological response, hormone receptor, protein involved in blood, transcription and apoptotic protein. In special preferred embodiments, the cannabinoid-related receptor is selected from a receptor class of the group consisting of hydrolase, transferase, oxidoreductase, signaling protein, cell adhesion protein, transport protein, nuclear receptor, metalloprotease, ligase, matrix metalloprotease, and transcription protein.

In special preferred embodiments of the invention, the multiomic analysis comprises cell profiling of the sample, preferably using a multiplexed immunofluorescence method. In special preferred embodiments, the multiplexed immunofluorescence method comprises a detection, optionally a quantification, of a cannabinoid-related receptor. In special preferred embodiments, the cannabinoid-related receptor is selected from at least one cannabinoid-related receptor of the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA), PPARβ (also called PPARD) and PPARγ (also called PPARG). In special preferred embodiments, the cannabinoid-related receptor is CBR1, CBR2, TRPV1, TRPV2, TRPV3, TRPV4, TRPA1, TRPVA1, TRPM8, GPR3, GPR6, GPR12, GPR18, GPR23, GPR35, GPR55, GPR84, GPR92, GPR119, PPARα, PPARβ and/or PPARγ. In special preferred embodiments, the cannabinoid-related receptor is selected from a receptor class of the group consisting of hydrolase, transferase, oxidoreductase, signaling protein, cell adhesion protein, transport protein, nuclear receptor, metalloprotease, ligase, matrix metalloprotease, flavoprotein, cell cycle related protein, lyase, isomerase, protein involved in immunological response, hormone receptor, protein involved in blood, transcription and apoptotic protein. In special preferred embodiments, the cannabinoid-related receptor is selected from a receptor class of the group consisting of hydrolase, transferase, oxidoreductase, signaling protein, cell adhesion protein, transport protein, nuclear receptor, metalloprotease, ligase, matrix metalloprotease, and transcription protein.

In preferred embodiments of the invention, the multiomic analysis comprises a cell composition analysis and/or a neighborhood analysis. The cell composition analysis preferably determines the proportion and density of cell types in the sample. Preferably, the cell composition analysis does not consider all cell types. Preferably, in said cell composition analysis the proportion and density of cell selected from the group of B cell, early germinal center B cell, germinal center B cell, classical dendritic cell type I, follicular dendritic cell, high endothelial macrophage, natural killer cell, plasmocytic dendritic cell, cytotoxic T cell, regulatory T cell, T follicular helper cell, T helper cell, plasma cell, classical dendritic cell type II, stromal cell, epithelial cell and lymphatic vessel is determined.

In preferred embodiments of the invention, the multiomic analysis comprises a gene expression analysis, preferably, wherein the gene expression analysis is a spatial gene expression analysis. Preferably the gene expression analysis comprises determining the over expression or under expression of genes. Preferably said genes are selected from genes related to HLA class II, genes related to cytokines, chemokines and cell signaling receptors, genes related to T cell and NK cell function, genes related to B cell function, genes related to myeloids and monocytes, genes related to cell growth and differentiation, cell structure, motility, metabolic genes, and/or cell cycle related genes. In preferred embodiments of the invention, the gene expression analysis comprises a transcriptome analysis and, optionally, a transcription factor analysis and/or a pathway analysis. In special preferred embodiments, the gene expression analysis comprises conducting RT-qPCR (also known as real-time quantitative PCR, or real time quantitative polymerase chain reaction). In special preferred embodiments of the invention, the genes (in the context of the gene expression analysis) are genes related to HLA class II, genes related to cytokines, chemokines and cell signaling receptors, genes related to T cell and NK cell function, genes related to B cell function, genes related to myeloids and monocytes, genes related to cell growth and differentiation, cell structure, motility, metabolic genes, cell cycle related genes and/or genes related to a cannabinoid-related receptor. In special preferred embodiments, the cannabinoid related receptor is a cannabinoid-related receptor as disclosed in the context of the multiplexed immunofluorescence method.

In preferred embodiments of the invention, the multiomics analysis comprises a cytokine expression profiling, preferably wherein the cytokine expression profiling comprises conducting a multiplex ELISA.

In further preferred embodiments of the invention, the cytokine expression profiling comprises a detection, optionally a quantification, of at least one secretory cytokine selected from the group consisting of EGF, FGF, VEGF, TNF and Interleukins. In further special preferred embodiments, the cytokine expression profiling comprises a detection, optionally a quantification, of at least one secretory cytokine selected from the group consisting of EGF, FGF, VEGF, TNF, IFN, TGF, TRAIL, SCF, MSP, CD, OSM, BBL, GITRI, LIGHT, OX, TALL, TWEAK, TRANCE and Interleukins. In special preferred embodiments, the cytokine expression profiling comprises a detection, optionally a quantification, of at least one secretory cytokine selected from the structural families of four-α-helix bundle (preferably selected from the group of IL-2 subfamily, IFN subfamily and IL-10 subfamily), IL-1, cysteine knot cytokines and IL-17 family. In special preferred embodiments, the cytokine expression profiling comprises a detection, optionally a quantification, of at least one secretory cytokine selected from the group of EGF, FGF, VEGF, IL-1α, IL-1β, IL-1RA, IL-18, Common g chain (CD132), IL-2, IL-4, IL-7, IL-9, IL-13, IL-15, Common b chain (CD131), IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, and MSP.

In preferred embodiments of the invention, the multiomics analysis comprises a protein phosphorylation analysis, preferably, wherein the protein phosphorylation analysis comprises conducting a multiplex ELISA. Preferably, the protein phosphorylation analysis comprises a detection, optionally a quantification, of the phosphorylation of at least one intracellular signaling protein selected from the group consisting of MEK1, ERK1/2, AKT, FAK1, CHK2, JUN, CREB1, EGFR, RB, GSK3, HSP27, P38, IκB, NF-κB, mTOR, RSK1, SMAD3, AKT1S1, CHK2, MARCKS, p70S6K, LCK, PI3K, P53, PTN11, NRF2, STAT1, STAT3, STAT6, SRC and/or WNT. In special preferred embodiments, the protein phosphorylation analysis comprises a detection, optionally a quantification, of the phosphorylation of at least one intracellular signaling protein selected from the group consisting of MEK1/2, ERK1/2, P38, JUN, CREB, GSK3, STAT3, AKT, mTOR, AKT1S1, MARCKS, IKBA, SMAD3, HSP27 and P53.

In preferred embodiments of the invention, the computational analysis comprises correlating the predetermined chemical profile with the therapeutic efficacy of the composition comprising at least one cannabinoid to create a central database. Preferably, the central database is created by merging the data using the Constance Data Miner software (KNIME). In special preferred embodiments, the computational analysis is also employed for patient stratification and profiling using available biomarker analysis. Preferably, the central database is created by merging multiple data sources using big data analysis tools and libraries such as the Constance Data Miner software (KNIME).

In preferred embodiments of the invention, the computational analysis comprises the use of an in silico model, preferably wherein the in silico model describes metabolic pathways and/or gene expression of a subject (such as of a subject suffering from cancer) or preferably of a cell (such as of a cancer cell), when exposing the subject or preferably the cell to the composition comprising at least one cannabinoid. In special preferred embodiments of the invention, the in silico model describes docking scores or energy calculations of cannabinoids on a potential therapeutic cannabinoid target or target ensemble, metabolic pathways and/or gene expression of a subject (such as of a subject suffering from cancer) or preferably of a cell (such as of a cancer cell), when exposing the subject or preferably the cell to the composition comprising at least one cannabinoid. In special preferred embodiments of the invention, the in silico model describes docking scores or energy calculations of cannabinoids on a potential therapeutic cannabinoid target or target ensemble of a subject, when exposing the subject to the composition comprising at least one cannabinoid. In special preferred embodiments of the invention, the subject is a cancer subject. In special preferred embodiments of the invention, the in silico model describes docking scores or energy calculations of cannabinoids on a potential therapeutic cannabinoid target or target ensemble of a cell, when exposing the cell to the composition comprising at least one cannabinoid. In special preferred embodiments, the cell is a cancer cell.

In special preferred embodiments of the invention, the potential therapeutic cannabinoid target is selected from the group consisting of potential therapeutic cannabinoid targets with the 5-letter target names 2c0tA, 4lucB, 4zt1A, 4lv6B, 1flsA, 3f16A, 2d1jA, 2jt5A, 4zt1B, 5v6vB, 5hl1D, 3mo2A, 3cemA, 3wd9A, 2aykA, 5uqeD, 5i94D, 4g1mB, 5nawA, 5fi6B, 5i94A, 4qfcA, 5hl1B, 3aykA, 4yiaB, 1exvA, 5x16A, 5dy4A, 4bghA, 4nvqB, 4wj8D, 3rpyA, 3es3A, 5qd1A, 5jypA, 3lfnA, 4zteB, 5u3sA, 6hoyA, 5nb6A, 1mqbA, 3vbxA, 5mgnA, 1p2aA, 6f2lA, 3cehA, 3k23A, 6qchA, 6pmeB, 5v6vA, 3wblA, 5nb7A, 1t5aC, 3r7uA, 5mfzA, 5i94C, 5mf6A, 4ey5B, 4xifA, 5ml4B, 1zjhA, 2wnsB, 2prgA, 3nxyA, 5i94B, 3ghvA, 6hj2A, 6tu1A, 6opiA, 4bb6A, 1ohkA, 3uo9B, 1xurB, 6ylkA, 5fbeA, 1mzdA, 5qcrA, 5k4jA, 5qikA, 5tccA, 2r3jA, 5uqeB, 3rmfA, 4ek4A, 5ztnA, 2b58A, 2wnsA, 3r1sA, 6qceA, 1w51A, 6vnvA, 5ayyG, 1wywA, 6s4nD, 3gjxB, 2c6iA, 1dfpA, 5otlB, 5fbiA and/or 4hdaA. In special preferred embodiments, in the context of these 5-letter target names (for example “3f16A”), the first 4 letters of the 5-letter target name indicate the protein database ID (PDB ID) under which the protein that describes the potential therapeutic cannabinoid target can be found in the RCSB PDB Database (https://www.rcsb.org/, dated 27 Jun. 2023) and the last letter of the 5-letter target name indicates the chain of the protein that describes the potential therapeutic cannabinoid target. For example, in this context the potential therapeutic cannabinoid target “3f16A” refers to the chain “A” of the protein that is described by the protein database entry “3F16”. Table 1 gives further details on the 5-letter target names along with the respective PDB ID, chain, classification and description.

TABLE 1
Information on potential therapeutic cannabinoid targets of the special preferred embodiments

| Target | PDB | | | |
|---|---|---|---|---|
| Name | ID | Chain | Classification | Descriptor |
| 2c0tA | 2c0t | A | TRANSFERASE | TYROSINE-PROTEIN KINASE HCK (E.C.2.7.1.112) |
| 4lucB | 4luc | B | SIGNALING | GTPase KRas |
| | | | PROTEIN/INHIBITOR | |
| 4zt1A | 4zt1 | A | CELL ADHESION | Cadherin-3 |
| 4lv6B | 4lv6 | B | SIGNALING | GTPase KRas |
| | | | PROTEIN/INHIBITOR | |
| 1flsA | 1fls | A | HYDROLASE | SOLUTION STRUCTURE OF THE CATALYTIC |
| | | | | FRAGMENT OF HUMAN COLLAGENASE-3 (MMP-13) |
| | | | | COMPLEXED WITH A HYDROXAMIC ACID INHIBITOR |
| 3f16A | 3f16 | A | HYDROLASE | Crystal structure of the catalytic domain of human |
| | | | | MMP12 complexed with the inhibitor (R)-N-(3-hydroxy- |
| | | | | 1-nitroso-1-oxopropan-2-yl)-4- |
| | | | | methoxybenzenesulfonamide |
| 2d1jA | 2d1j | A | HYDROLASE | Coagulation factor X, heavy chain (E.C.3.4.21.6), |
| | | | | Coagulation factor X, light chain (E.C.3.4.21.6) |
| 2jt5A | 2jt5 | A | HYDROLASE | Stromelysin-1 (E.C.3.4.24.17) |
| 4zt1B | 4zt1 | B | CELL ADHESION | Cadherin-3 |
| 5v6vB | 5v6v | B | HYDROLASE/HYDROLASE | GTPase KRas |
| | | | INHIBITOR | |
| 5hl1D | 5hl1 | D | HYDROLASE/HYDROLASE | Glutaminase kidney isoform, mitochondrial |
| | | | INHIBITOR | (E.C.3.5.1.2) |
| 3mo2A | 3mo2 | A | TRANSFERASE | Histone-lysine N-methyltransferase, H3 lysine-9 |
| | | | | specific 5 (E.C.2.1.1.43) |
| 3cemA | 3cem | A | TRANSFERASE | Glycogen phosphorylase (E.C.2.4.1.1) |
| 3wd9A | 3wd9 | A | HYDROLASE/HYDROLASE | cAMP-specific 3′,5′-cyclic phosphodiesterase 4B |
| | | | INHIBITOR | (E.C.3.1.4.17) |
| 2aykA | 2ayk | A | METALLOPROTEASE | INHIBITOR-FREE CATALYTIC FRAGMENT OF HUMAN |
| | | | | FIBROBLAST COLLAGENASE, NMR, MINIMIZED |
| | | | | AVERAGE STRUCTURE |
| 5uqeD | 5uge | D | HYDROLASE | Glutaminase liver isoform, mitochondrial (E.C.3.5.1.2) |
| 5i94D | 5i94 | D | HYDROLASE/HYDROLASE | Glutaminase kidney isoform, mitochondrial |
| | | | INHIBITOR | (E.C.3.5.1.2) |
| 4g1mB | 4g1m | B | PROTEIN BINDING | Integrin alpha-V, Integrin beta-3 |
| 5nawA | 5naw | A | HYDROLASE | Complement factor D (E.C.3.4.21.46) |
| 5fi6B | 5fi6 | B | HYDROLASE/HYDROLASE | human glutaminase C |
| | | | INHIBITOR | |
| 5i94A | 5i94 | A | HYDROLASE/HYDROLASE | Glutaminase kidney isoform, mitochondrial |
| | | | INHIBITOR | (E.C.3.5.1.2) |
| 4qfcA | 4qfc | A | OXIDOREDUCTASE/ | D-amino-acid oxidase (E.C.1.4.3.3) |
| | | | OXIDOREDUCTASE | |
| | | | INHIBITOR | |
| 5hl1B | 5hl1 | B | HYDROLASE/HYDROLASE | Glutaminase kidney isoform, mitochondrial |
| | | | INHIBITOR | (E.C.3.5.1.2) |
| 3aykA | 3ayk | A | MATRIX | COLLAGENASE (E.C.3.4.24.7) |
| | | | METALLOPROTEINASE | |
| 4yiaB | 4yia | B | SIGNALING PROTEIN | Thyroxine binding globulin |
| 1exvA | 1exv | A | TRANSFERASE | GLYCOGEN PHOSPHORYLASE, PYRIDOXAL-5′- |
| | | | | PHOSPHATE, 1-{2-[5-CHLORO-1H-INDOLE-2- |
| | | | | CARBONYL)-AMINO]-3-PHEYNYL-PROPIONYL}- |
| | | | | AZETIDINE-3-CARBOXYLIC ACID |
| 5x16A | 5x16 | A | HYDROLASE | NAD-dependent protein deacetylase sirtuin-6 |
| | | | | (E.C.3.5.1.—) |
| 5dy4A | 5dy4 | A | HYDROLASE | NAD-dependent protein deacetylase sirtuin-2 |
| | | | | (E.C.3.5.1.—) |
| 4bghA | 4bgh | A | TRANSFERASE | CYCLIN-DEPENDENT KINASE 2 (E.C.2.7.11.22) |
| 4nvqB | 4nvq | B | TRANSFERASE/ | Histone-lysine N-methyltransferase EHMT2 (E.C.2.1.1.—, |
| | | | TRANSFERASE INHIBITOR | 2.1.1.43) |
| 4wj8D | 4wj8 | D | TRANSFERASE | Pyruvate kinase isozymes M1/M2 (E.C.2.7.1.40) |
| 3rpyA | 3rpy | A | TRANSFERASE/ | Cyclin-dependent kinase 2 (E.C.2.7.11.22) |
| | | | TRANSFERASE INHIBITOR | |
| 3es3A | 3es3 | A | OXIDOREDUCTASE | Directing Noble Metal Ion Chemistry within a Designed |
| | | | | Ferritin Protein. The Complex with Gold ions. Ferritin |
| | | | | H8-H9x Mutant |
| 5qd1A | 5qd1 | A | HYDROLASE | Beta-secretase 1 (E.C.3.4.23.46) |
| 5jypA | 5jyp | A | HYDROLASE | Glutaminase kidney isoform, mitochondrial |
| | | | | (E.C.3.5.1.2) |
| 3lfnA | 3lfn | A | TRANSFERASE | Cell division protein kinase 2 (E.C.2.7.11.22) |
| 4zteB | 4zte | B | CELL ADHESION | Cadherin-3 |
| 5u3sA | 5u3s | A | PROTEIN | Peroxisome proliferator-activated receptor delta |
| | | | BINDING/ACTIVATOR | |
| 6hoyA | 6hoy | A | HYDROLASE | NAD-dependent protein deacetylase sirtuin-6 |
| | | | | (E.C.3.5.1.—) |
| 5nb6A | 5nb6 | A | HYDROLASE | Complement factor D (E.C.3.4.21.46) |
| 1mqbA | 1mqb | A | TRANSFERASE | Ephrin type-A receptor 2 (E.C.2.7.1.112) |
| 3vbxA | 3vbx | A | TRANSFERASE/ | Serine/threonine-protein kinase pim-1 (E.C.2.7.11.1) |
| | | | TRANSFERASE INHIBITOR | |
| 5mgnA | 5mgn | A | HYDROLASE | Human SIRT6 (E.C.3.5.1.17) |
| 1p2aA | 1p2a | A | TRANSFERASE | Cell division protein kinase 2, 5-[(2- |
| | | | | AMINOETHYL)AMINO]-6-FLUORO-3-(1H-PYRROL-2- |
| | | | | YL)BENZO[CD]INDOL-2(1H)-ONE |
| 6f2lA | 6f2l | A | TRANSCRIPTION | Peroxisome proliferator-activated receptor gamma |
| 3cehA | 3ceh | A | TRANSFERASE | Glycogen phosphorylase (E.C.2.4.1.1) |
| 3k23A | 3k23 | A | TRANSCRIPTION | Glucocorticoid Receptor with Bound D-prolinamide 11 |
| 6qchA | 6qch | A | HYDROLASE | NAD-dependent protein deacetylase sirtuin-6 |
| | | | | (E.C.3.5.1.—) |
| 6pmeB | 6pme | B | TRANSFERASE | High affinity nerve growth factor receptor |
| | | | | (E.C.2.7.10.1) |
| 5v6vA | 5v6v | A | HYDROLASE/HYDROLASE | GTPase KRas |
| | | | INHIBITOR | |
| 3wblA | 3wbl | A | TRANSFERASE/ | Cyclin-dependent kinase 2 (E.C.2.7.11.22) |
| | | | TRANSFERASE INHIBITOR | |
| 5nb7A | 5nb7 | A | HYDROLASE | Complement factor D (E.C.3.4.21.46) |
| 1t5aC | 1t5a | C | TRANSFERASE | Pyruvate kinase, M2 isozyme (E.C.2.7.1.40) |
| 3r7uA | 3r7u | A | TRANSFERASE/ | Cyclin-dependent kinase 2 (E.C.2.7.11.22) |
| | | | TRANSFERASE INHIBITOR | |
| 5mfzA | 5mfz | A | HYDROLASE | Human SIRT6 (E.C.3.5.1.17) |
| 5i94C | 5i94 | C | HYDROLASE/HYDROLASE | Glutaminase kidney isoform, mitochondrial |
| | | | INHIBITOR | (E.C.3.5.1.2) |
| 5mf6A | 5mf6 | A | HYDROLASE | Human SIRT6 (E.C.3.5.1.17) |
| 4ey5B | 4ey5 | B | HYDROLASE/HYDROLASE | Acetylcholinesterase (E.C.3.1.1.7) |
| | | | INHIBITOR | |
| 4xifA | 4xif | A | TRANSFERASE | Human O-GlcNAc transferase (E.C.2.4.1.255), VAL- |
| | | | | THR-PRO-VAL-SER-THR-ALA-ALA |
| 5ml4B | 5ml4 | B | LIPID BINDING | Retinal rod rhodopsin-sensitive cGMP 3′,5′-cyclic |
| | | | PROTEIN | phosphodiesterase subunit delta |
| 1zjhA | 1zjh | A | TRANSFERASE | Pyruvate kinase, isozymes M1/M2 (E.C.2.7.1.40) |
| 2wnsB | 2wns | B | TRANSFERASE | OROTATE PHOSPHORIBOSYLTRANSFERASE |
| | | | | (E.C.2.4.2.10) |
| 2prgA | 2prg | A | COMPLEX | PEROXISOME PROLIFERATOR ACTIVATED RECEPTOR |
| | | | (THIAZOLIDINEDIONE/ | GAMMA, NUCLEAR RECEPTOR COACTIVATOR SRC-1, |
| | | | RECEPTOR) | 2,4-THIAZOLIDIINEDIONE, 5-[[4-[2-(METHYL-2- |
| | | | | PYRIDINYLAMINO)ETHOXY]PHENYL]METHYL]-(9CL) |
| 3nxyA | 3nxy | A | OXIDOREDUCTASE | Dihydrofolate reductase (E.C.1.5.1.3) |
| 5i94B | 5i94 | B | HYDROLASE/HYDROLASE | Glutaminase kidney isoform, mitochondrial |
| | | | INHIBITOR | (E.C.3.5.1.2) |
| 3ghvA | 3ghv | A | OXIDOREDUCTASE | Human dihydrofolate reductase Q35K/N64F double |
| | | | | mutant inhibitor complex |
| 6hj2A | 6hj2 | A | NUCLEAR PROTEIN | Crystal structure of hPXR in complex with dabrafenib |
| 6tu1A | 6tu1 | A | TRANSFERASE | CREB-binding protein (E.C.2.3.1.48) |
| 6opiA | 6opi | A | TRANSFERASE | Mitogen-activated protein kinase 1 (E.C.2.7.11.24) |
| 4bb6A | 4bb6 | A | OXIDOREDUCTASE | CORTICOSTEROID 11-BETA-DEHYDROGENASE |
| | | | | ISOZYME 1 (E.C.1.1.1.146) |
| 1ohkA | 1ohk | A | OXIDOREDUCTASE | DIHYDROFOLATE REDUCTASE, NADPH DIHYDRO- |
| | | | | NICOTINAMIDE-ADENINE-DINUCLEOTIDE |
| | | | | PHOSPHATE, N-(4-CARBOXY-4-{4-[(2,4-DIAMINO- |
| | | | | PTERIDIN-6-YLMETHYL)-AMINO]-BENZOYLAMINO}- |
| | | | | BUTYL)-PHTHALAMIC ACID |
| 3uo9B | 3uo9 | B | HYDROLASE/HYDROLASE | Glutaminase kidney isoform, mitochondrial |
| | | | INHIBITOR | (E.C.3.5.1.2) |
| 1xurB | 1xur | B | HYDROLASE | Collagenase 3 (E.C.3.4.24.—) |
| 6ylkA | 6ylk | A | TRANSFERASE | Cyclin-dependent kinase 2 (E.C.2.7.11.22) |
| 5fbeA | 5fbe | A | HYDROLASE | Kallikrein-7 (E.C.3.4.21.117) |
| 1mzdA | 1mzd | A | HYDROLASE | crystal structure of human pro-granzyme K |
| 5qcrA | 5qcr | A | HYDROLASE | Beta-secretase 1 (E.C.3.4.23.46) |
| 5k4jA | 5k4j | A | TRANSFERASE/ | Cyclin-dependent kinase 2 (E.C.2.7.11.22) |
| | | | TRANSFERASE INHIBITOR | |
| 5qikA | 5qik | A | TRANSFERASE/ | TGF-beta receptor type-1 (E.C.2.7.11.30) |
| | | | TRANSFERASE INHIBITOR | |
| 5tccA | 5tcc | A | HYDROLASE/HYDROLASE | Complement factor D (E.C.3.4.21.46) |
| | | | INHIBITOR | |
| 2r3jA | 2r3j | A | TRANSFERASE | Cell division protein kinase 2 (E.C.2.7.11.22) |
| 5uqeB | 5uge | B | HYDROLASE | Glutaminase liver isoform, mitochondrial (E.C.3.5.1.2) |
| 3rmfA | 3rmf | A | TRANSFERASE/ | Cyclin-dependent kinase 2 (E.C.2.7.11.22) |
| | | | TRANSFERASE INHIBITOR | |
| 4ek4A | 4ek4 | A | TRANSFERASE/ | Cyclin-dependent kinase 2 (E.C.2.7.11.22) |
| | | | TRANSFERASE INHIBITOR | |
| 5ztnA | 5ztn | A | TRANSFERASE/ | Dual specificity tyrosine-phosphorylation-regulated |
| | | | TRANSFERASE INHIBITOR | kinase 2 (E.C.2.7.12.1) |
| 2b58A | 2b58 | A | TRANSFERASE | Diamine acetyltransferase 1 (E.C.2.3.1.57) |
| 2wnsA | 2wns | A | TRANSFERASE | OROTATE PHOSPHORIBOSYLTRANSFERASE |
| | | | | (E.C.2.4.2.10) |
| 3r1sA | 3r1s | A | TRANSFERASE/ | Cyclin-dependent kinase 2 (E.C.2.7.11.22) |
| | | | TRANSFERASE INHIBITOR | |
| 6qceA | 6qce | A | HYDROLASE | NAD-dependent protein deacetylase sirtuin-6 |
| | | | | (E.C.3.5.1.—) |
| 1w51A | 1w51 | A | HYDROLASE/INHIBITOR | BETA-SECRETASE 1 (E.C.3.4.23.46) |
| 6vnvA | 6vnv | A | TRANSFERASE/INHIBITOR | Non-receptor tyrosine-protein kinase TYK2 |
| | | | | (E.C.2.7.10.2) |
| 5ayyG | 5ayy | G | TRANSFERASE | NICOTINATE-NUCLEOTIDE PYROPHOSPHORYLASE |
| | | | | [CARBOXYLATING] (E.C.2.4.2.19) |
| 1wywA | 1wyw | A | HYDROLASE | G/T mismatch-specific thymine DNA glycosylase |
| | | | | (E.C.3.2.2.—), Ubiquitin-like protein SMT3C |
| 6s4nD | 6s4n | D | NUCLEAR PROTEIN | Oxysterols receptor LXR-beta |
| 3gjxB | 3gjx | B | PROTEIN TRANSPORT | Crystal Structure of the Nuclear Export Complex |
| | | | | CRM1-Snurportin1-RanGTP |
| 2c6iA | 2c6i | A | TRANSFERASE | CELL DIVISION PROTEIN KINASE 2 (E.C.2.7.1.37) |
| 1dfpA | 1dfp | A | SERINE PROTEASE | FACTOR D, DIISOPROPYLPHOSPHONO GROUP |
| 5otlB | 5otl | B | TRANSFERASE | Casein kinase II subunit alpha (E.C.2.7.11.1) |
| 5fbiA | 5fbi | A | HYDROLASE | Kallikrein-7 (E.C.3.4.21.117) |
| 4hdaA | 4hda | A | HYDROLASE/HYDROLASE | NAD-dependent protein deacylase sirtuin-5, |
| | | | ACTIVATOR | mitochondrial (E.C.3.5.1.—), Fluor-de-Lys peptide |

In special preferred embodiments, the in silico model is composed as mathematically modelled logical regulatory network or as a deep-learning architecture-type network, both representing a biological pathway and its responses to applying the composition comprising at least one cannabinoid. In special preferred embodiments, the in silico model is constructed using a network analysis and deep learning framework such as FALCON, PyTorch, Keras and/or TensorFlow. In special preferred embodiments of the invention, the biological pathway is a biological pathway in a cell.

In preferred embodiments of the invention, the computational analysis comprises creating and/or visualizing data networks, analyzing nodes, and/or performing cluster analyses. In special preferred embodiments of the invention, the computational analysis comprises creating and visualizing data networks, heat map charts, analyzing nodes, and/or performing cluster analyses.

Preferably, the creation and visualization of data networks, analyzing nodes, and performing cluster analyses is conducted using the Constance Data Miner software (KNIME) and Gephi. In special preferred embodiments, the computational analysis comprises conducting an inverse molecular docking analysis. In special preferred embodiments, inverse molecular docking analysis comprises evaluating data from a complete human proteome target space. In special preferred embodiments, the computational analysis comprises a creation of inverse molecular docking studies and a creation of inverse docking datasets and heatmaps along with a corresponding inverse molecular docking analysis and molecular clustering (also sometimes named reverse docking, or docking selectivity studies). In special preferred embodiments, the inverse molecular docking analysis includes a complete human proteome target space. This target space allows to identify cannabinoid or cannabinoid composition biological targets. Furthermore, the data of the inverse molecular docking analysis can in special preferred embodiments be used to study the biological effects of cannabinoid compositions and/or to select an appropriate cannabinoid composition, for example for therapy. In special preferred embodiments, this selection depends on the biomarker, the biomarker expression and/or a cell viability analysis. In special preferred embodiments, the inverse molecular docking analysis is performed with a docking software. In special preferred embodiments, the inverse molecular docking analysis may be performed with any suitable docking algorithm, preferably with a molecular docking algorithm, more preferably the ProBiS-Dock docking algorithm. In special preferred embodiments, the inverse molecular docking analysis is performed with a docking software that comprises a suitable docking algorithm, such as a molecular docking algorithm. In special preferred embodiments, the molecular docking analysis is performed with a docking software that comprises a molecular docking algorithm, preferably the ProBis-Dock docking algorithm. In special preferred embodiments, the inverse molecular docking analysis is performed with a docking software that comprises the ProBiS-Dock docking algorithm. Further information on the ProBiS-Dock docking algorithm can be found in Konc et al (16) and http://insilab.org/probisdock/(27 Jun. 2023).

In preferred embodiments of the invention, the computational analysis comprises the creation of a database of potential therapeutic cannabinoid targets, preferably wherein the database of potential therapeutic cannabinoid targets is created by the use of in silico target prediction methods. In special preferred embodiments, the database of potential therapeutic cannabinoid targets comprises the complete human proteome as experimentally available from RCSB PDB, Uniprot, AlfaFold and/or ESM Metagenomic Atlas databases. In special preferred embodiments, the database of potential therapeutic cannabinoid targets comprises AKT, CREB1, GSK3, Hsp27, IKBA, MEK1/2, mTOR, p38 MAPK, p53, RSK1, Smad3, AKT1S1, CHK2, cJUN, EGFR, p44/42 MAPK (ERK1/2), MARCKS, NF-κB, STAT3, PTN11, FAK, JNK, p70S6K and/or GAPDH, more preferably the database of potential therapeutic cannabinoid targets comprises AKT, CREB1, GSK3, Hsp27, IKBA, MEK1/2, mTOR, p38 MAPK, p53, RSK1, Smad3, AKT1S1, CHK2, cJUN, EGFR, p44/42 MAPK (ERK1/2), MARCKS, NF-κB, STAT3, PTN11, FAK, JNK, p70S6K and/or GAPDH.

In preferred embodiments of the invention, the in silico target prediction methods are selected from SwissTarget, BioGRID, canCAR and/or the CaNDis web server. In special preferred embodiments, the creation of a database of potential therapeutic cannabinoid targets comprises a target comparison with protein structures from RCSB PDB, AlfaFold and/or ESM Metagenomic Atlas databases.

In preferred embodiments of the invention, the computational analysis comprises steps for identifying therapeutic cannabinoid targets comprising:
Matching of a potential therapeutic cannabinoid target with its gene data, preferably by using the Enrichr toolset and/or
Matching a potential therapeutic cannabinoid target by its presence in key biological pathways, preferably by using the KEGG database.

In special preferred embodiments of the invention, the computational analysis comprises steps for identifying potential therapeutic cannabinoid targets comprising:
Matching of a potential therapeutic cannabinoid target with its gene data, preferably by using the Enrichr toolset,
Matching a potential therapeutic cannabinoid target by its presence in key biological pathways, preferably by using the KEGG database, and/or
Matching of a potential therapeutic cannabinoid target with its gene and expression data by using deep-learning type models, using Pythorch, KERAS and/or TensorFlow libraries.

In preferred embodiments of the invention, the potential therapeutic cannabinoid targets are linked to common interaction partners in the protein-protein interaction String database, optionally wherein additional therapeutic targets and/or mechanisms of observed adverse drug effects are identified.

In preferred embodiments of the invention, the potential therapeutic cannabinoid targets are in the form of experimental crystal complexes such as in the RCSB PDB Database. In further special preferred embodiments of the invention, the potential therapeutic cannabinoid targets are in the form of experimental crystal complexes such as in the RCSB PDB Database, the AlfaFold and/or the ESM Metagenomic Atlas database.

In preferred embodiments of the invention, the potential therapeutic cannabinoid targets are in the form of homology models such as in SwissModel, Modeller and/or Yasara.

In preferred embodiments of the invention, the method further comprises studying the interaction of cannabinoids, preferably cannabinoids of the composition comprising at least one cannabinoid, and their metabolites with the potential therapeutic cannabinoid targets, preferably at an atomistic level. In special preferred embodiments of the invention, the method includes inverse molecular docking analysis and creation of inverse molecular docking fingerprints, which are further subjected to machine learning and/or statistical analysis to identify key cannabinoid-target interactions.

In preferred embodiments of the invention, the interaction of the cannabinoids and/or their metabolites with their potential therapeutic cannabinoid targets is evaluated by using direct methods, preferably inverse molecular docking, and/or by constructing drug-target interaction fingerprints, maps and/or structure-activity relationships. In special preferred embodiments of the invention, the direct methods are structure-based methods, ligand-based methods and/or consensus methods. In special preferred embodiments of the invention, the direct methods are structure-based methods, ligand-based methods and consensus methods

In preferred embodiments of the invention, the computational analysis is based on machine learning. In special preferred embodiments of the invention, the computational analysis is based on machine learning and/or statistical analysis. In special preferred embodiments of the invention, the computational analysis is based on machine learning and statistical analysis.

In preferred embodiments of the invention, the method for identifying and/or optimizing a therapeutic composition against cancer comprising at least one cannabinoid comprises setting up mechanistic models of action of individual cannabinoids and/or combinations of cannabinoids, preferably wherein the mechanistic models of action of individual cannabinoids and/or combinations of cannabinoids are suitable to rationally set effective therapy for selected cancer patients.

In special preferred embodiments of the invention, the method for identifying a therapeutic composition against cancer comprising at least one cannabinoid comprises setting up mechanistic models of action of individual cannabinoids and/or combinations of cannabinoids, preferably wherein the mechanistic models of action of individual cannabinoids and/or combinations of cannabinoids are suitable to rationally set effective therapy for selected cancer patients. In special preferred embodiments, said models comprise mathematically modelled logical regulatory networks or deep-learning architecture-type networks, representing biological pathways and responses on the application of the composition comprising at least one cannabinoid.

In a second aspect, the invention pertains to a therapeutic composition against cancer comprising at least one cannabinoid.

In preferred embodiments of the invention, the therapeutic composition against cancer comprises at least one pharmaceutically acceptable carrier and/or excipient.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, solvent, diluent, encapsulating material, surfactant, preservative, stabilizer, anti-oxidant, coloring agent, or formulation auxiliary of any type.

Preferably, the pharmaceutically acceptable carrier is selected from at least one component of the group consisting of: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption such as cyclodextrin, hydroxypropyl-cyclodextrin, PEG400, PEG200.

In preferred embodiments of the invention, the therapeutic composition against cancer comprising at least one cannabinoid wherein the cannabinoid is a phytocannabinoid (pCB). In special preferred embodiments of the invention, the cannabinoid is a phytocannabinoid (pCB).

In preferred embodiments of the invention, the at least one phytocannabinoid is selected from the group consisting of (−)-Δ9-trans-tetrahydrocannabinol type (also referred to as Δ9-THC-type), (−)-48-trans-tetrahydrocannabinol type (also referred to as Δ8-THC-type), cannabigerol type (also referred to as CBG-type), cannabichromene type (also referred to as CBC-type), cannabidiol type (also referred to as CBD-type), cannabinodiol types (also referred to as CBND-type), cannabielsoin type (also referred to as CBE-type), cannabicyclol type (also referred to as CBL-type), cannabinol type (also referred to as CBN-type), cannabitriol type (also referred to as CBT-type) and miscellaneous type.

In preferred embodiments of the invention, the Δ9-THC-type is selected from the group consisting of Δ⁹-tetrahydrocannabinol (Δ⁹-THC-C₅, also referred to as Δ9THC, THC, or THC-d9), Δ⁹-tetrahydrocannabinol-C₄ (Δ9-THC-C₄), Δ9-tetrahydrocannabivarin (Δ⁹-THCV-C₃, also referred to as THCV), Δ⁹-tetrahydrocannabiorcol (Δ⁹-THCO-C₁), Δ⁹-tetrahydrocannabinolic acid A (Δ⁹-THCA-C₅ A also referred to THCA), Δ⁹-tetrahydrocannabinolic acid B (Δ⁹-THCA-C₅ B), Δ⁹-tetrahydrocannabinolic acid-C₄ A (Δ⁹-THCA-C₄ A), Δ⁹-tetrahydrocannabinolic acid-C₄ B (Δ⁹-THCA-C₄ B), Δ⁹-tetrahydrocannabivarinic acid A (Δ⁹-THCVA-C₃ A, also referred to as THCVA), Δ⁹-tetrahydrocannabiorcolic acid A (Δ⁹-THCOA-C₁ A), Δ⁹-tetrahydrocannabiorcolic acid B (Δ⁹-THCOA-C₁ B) and (−)-(6aS,10aR)-Δ9-tetrahydrocannabinol ((−)-cis-Δ⁹-THC-C₅).

In preferred embodiments of the invention, the Δ8-THC-type is selected from the group consisting of (−)-Δ⁸-trans-(6aR,10aR)-Δ⁸-tetrahydrocannabinol (Δ⁸-THC-C5, also referred to as Δ8THC) and (−)-Δ⁸-trans-(6aR,10aR)-tetrahydrocannabinolic acid A (Δ⁸-THCA-C₅ A).

In preferred embodiments of the invention, the CBND-type is selected from the group consisting of cannabinodiol (CBND-C₅) and cannabinodivarin (CBND-C₃).

In preferred embodiments of the invention, the CBD-type is selected from the group consisting of (−)-cannabidiol (CBD-C₅, also referred to as CBD), cannabidiol momomethyl ether CBDM-C₅, cannabidiol-C₄ (CBD-C₄), (−)-cannabidivarin (CBDV-C₃, also referred to as CBDV), cannabidiorcol (CBD-C₁), cannabidiolic acid (CBDA-C₅, also referred to as CBDA) and cannabidivarinic acid (CBDVA-C₃, also referred to as CBDVA)).

In preferred embodiments of the invention, the CBC-type is selected from the group consisting of (±)-cannabichromene (CBC-C₅, also referred to as CBC), (±)-cannabichromenic acid A (CBCA-C₅ A, also referred to as CBCA), (±)-cannabivarichromene (also known as (±)-cannabichromevarin, CBCV-C₃) and (±)-cannabichromevarinic acid A (CBCVA-C₃ A).

In preferred embodiments of the invention, the CBN-type is selected from the group consisting of cannabinol (CBN-C₅, also referred to as CBN), cannabinol-C₄ (CBN-C₄), cannabivarin (CBN-C₃), cannabinol-C₂ (CBN-C₂), cannabiorcol (CBN-C₁), cannabinolic acid A (CBNA-C₅ A) and cannabinol methyl ether (CBNM-C₅).

In preferred embodiments of the invention, the CBG-type is selected from the group consisting of cannabigerol ((E)-CBG-C₅, also referred to as CBG), cannabigerol monomethyl ether ((E)-CBGM-C₅ A), cannabinerolic acid A ((Z)-CBGA-C₅ A), cannabigerovarin ((E)-CBGV-C₃), cannabigerolic acid A ((E)-CBGA-C₅ A, also referred to as CBGA), cannabigerolic acid A monomethyl ether ((E)-CBGAM-C₅ A) and cannabigerovarinic acid A ((E)-CBGVA-C₃ A).

In preferred embodiments of the invention, the CBT-type is selected from the group consisting of (−)-(9R,10R)-trans-cannabitriol ((−)-trans-CBT-C₅), (+)-(9S,10S)-cannabitriol ((+)-trans-CBT-C₅), (±)-(9R,10S/9S,10R)-cannabitriol ((±)-cis-CBT-C₅), (−)-(9R,10R)-trans-10-O-ethyl-cannabitriol ((−)-trans-CBT-OEt-C₅), (±)-(9R,10R/9S,10S)-cannabitriol-C₃ ((±)-trans-CBT-C₃), 8,9-dihydroxy-Δ^{6a(10a)}-tetrahydrocannabinol (8,9-Di-OH-CBT-C₅), cannabidiolic acid A cannabitriol ester (CBDA-C₅ 9-OH-CBT-C₅ ester), (−)-(6aR,9S,10S,10aR)-9,10-dihydroxy-hexahydrocannabinol, (also known as cannabiripsol, cannabiripsol-C₅), (−)-6a,7,10a-trihydroxy-Δ⁹-tetrahydrocannabinol ((−)-cannabitetrol) and 10-oxo-Δ^{6a(10a)}-tetrahydrocannabinol (OTHC).

In preferred embodiments of the invention, the CBE-type is selected from the group consisting of (5aS,6S,9R,9aR)-cannabielsoin (CBE-C₅), (5aS,6S,9R,9aR)—C₃-cannabielsoin (CBE-C₃), (5aS,6S,9R,9aR)-cannabielsoic acid A (CBEA-C5 A), (5aS,6S,9R,9aR)-cannabielsoic acid B (CBEA-C₅ B), (5aS,6S,9R,9aR)—C₃-cannabielsoic acid B (CBEA-C₃ B), cannabiglendol-C₃ (OH-iso-HHCV-C₃), dehydrocannabifuran (DCBF-C₅) and cannabifuran (CBF-C₅).

In preferred embodiments of the invention, the CBL-type is selected from the group consisting of (±)-(1aS,3aR,8bR,8cR)-cannabicyclol (CBL-C₅, also referred to as CBL), (±)-(1aS,3aR,8bR,8cR)-cannabicyclolic acid A (CBLA-C₅ A, also referred to as CBLA) and (±)-(1aS,3aR,8bR,8cR)-cannabicyclovarin (CBLV-C₃).

In preferred embodiments of the invention, the miscellaneous type is selected from the group consisting of
isocannabinoids such as (−)-Δ⁷-trans-(1R,3R,6R)-isotetrahydrocannabinol, (±)-Δ⁷-1,2-cis-(1R,3R,6S/1S,3S,6R)-isotetrahydro-cannabivarin and (−)-Δ⁷-trans-(1R,3R,6R)-isotetrahydrocannabivarin,
cannabicitran-types such as cannabicitran, and
cannabichromanone-types (CBCN-type) such as cannabichromanone (CBCN-C₅), cannabichromanone-C₃ (CBCN-C₃) and cannabicoumaronone (CBCON-C₅).

In special preferred embodiments of the invention, the at least one cannabinoid is selected from the group consisting of Δ9-THCV-C3, Δ9-THCVA-C3 A, Δ9-THCA-C5 A, Δ9-THC-C5, Δ8-THC-C5, CBN-C5, CBL-C5, CBLA-C5 A, (E)-CBG-C5, (E)-CBGA-C5 A, CBD-C5, CBDV-C3, CBDVA-C3, CBDA-C5, CBC-C5, and CBCA-C5 A.

In special preferred embodiments of the invention, the at least one cannabinoid is CBN, CBG, CBD, CBC, Δ9-THC and/or Δ8-THC. In special preferred embodiments of the invention, the at least one cannabinoid is CBN, CBG, CBD and/or CBC.

In special preferred embodiments of the invention, the at least one cannabinoid is THCA, THCVA, CBLA, CBGA, CBDVA, and/or CBDA. In special preferred embodiments of the invention, the at least one cannabinoid is CBGA, THCA and/or CBLA, more preferably the at least one cannabinoid is THCA and/or CBLA, even more preferably the at least one cannabinoid is CBLA. In special preferred embodiments of the invention, the at least one cannabinoid is CBCA and/or THCV.

In preferred embodiments of the invention, the therapeutic composition against cancer comprising at least one cannabinoid is a therapeutic composition against cancer comprising at least one cannabinoid obtainable by the method of the other aspects of the invention. In this context, “obtainable” or “obtained” is meant to indicate that the therapeutic composition against cancer comprising at least one cannabinoid is a therapeutic composition against cancer comprising at least one cannabinoid that was identified and/or optimized using a method of the present invention.

In a third aspect, the invention pertains to a therapeutic composition against cancer comprising at least one cannabinoid, for use in the treatment of a condition or disease, preferably wherein the therapeutic composition against cancer comprising at least one cannabinoid is the therapeutic composition against cancer comprising at least one cannabinoid according to any other aspect of the invention.

In general, the invention provides a method for treating a patient suffering from cancer and/or a disease associated with cancer comprising the administration of the composition comprising at least one cannabinoid according to the invention. The term “patient” in the context of the invention is interchangeable with the term “subject”. Preferably the patient is a subject in need of such a treatment. The subject in preferred embodiments is a mammalian subject, preferably a human subject, suffering from cancer and/or a disease associated with cancer.

In preferred embodiments of the invention, the disease is cancer. Cancer in the context of the invention refers to any cancer, including any of acute lymphocytic cancer, acute myeloid leukemia, alveolar rhabdomyosarcoma, bone cancer, brain cancer, breast cancer, cancer of the anus, anal canal, or anorectum, cervical cancer, cancer of the eye, cancer of the intrahepatic bile duct, cancer of the joints, cancer of the head, cancer of the neck, gallbladder, or pleura, cancer of the nose, nasal cavity, or middle ear, cancer of the oral cavity, cancer of the vagina, cancer of the vulva, leukemia such as chronic lymphocytic leukemia, chronic myeloid cancer, colon cancer, colorectal cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid, cancer of the uterus, ureter cancer, and urinary bladder cancer. In preferred embodiments of the invention, the cancer is selected from the group consisting of lung cancer, colorectal cancer, thyroid cancer, cervical cancer, breast cancer, brain cancer, skin cancer and leukemia. In preferred embodiments of the invention, the skin cancer is a melanoma. In special preferred embodiments of the invention, the cancer is skin cancer. In special preferred embodiments of the invention, the cancer is colon cancer.

Preferably, the therapeutic composition against cancer comprising at least one cannabinoid is administered in an amount sufficient to achieve the therapeutic effect or to prolong the survival of the patient. The amount sufficient to achieve the therapeutic effect or to prolong the survival of the patient may be determined or estimated from suitable in vitro and/or cell culture assays. Furthermore, a dose can be formulated in animal models to achieve a desired concentration or titer. Such information can be used to determine useful doses in humans.

Preferably, the treatment comprises administration of the therapeutic composition against cancer comprising at least one cannabinoid. Preferably, the composition comprising at least one cannabinoid is administered by a route selected from oral, rectal, transmucosal, intestinal or parenteral delivery, including intramuscular, subcutaneous and intramedullary injections as well as intrathecal, direct intraventricular, intracardiac, intravenous, intraperitoneal, intranasal, or intraocular injection. In preferred embodiments, the therapeutic composition against cancer comprising at least one cannabinoid is administered in a local rather than systemic manner. Such a local administration may for example be conducted by injection of the of the therapeutic composition against cancer comprising at least one cannabinoid directly into a tissue region, preferably a tissue region that is comprising cancer cells, of a patient.

The therapeutic composition against cancer comprising at least one cannabinoid may be formulated depending on the route of administration. Preferably, the therapeutic composition against cancer comprising at least one cannabinoid is formulated in a solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

In preferred embodiments of the invention, the treatment targets the endocannabinoid system. In this context, the treatment preferably influences (such as increases or decreases) the expression of at least one receptor selected from the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA), PPARβ (also called PPARD) and PPARγ (also called PPARG). In this context, the treatment in special preferred embodiments preferably influences (such as increases or decreases, preferably increases) the expression of at least one receptor selected from the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA), PPARβ (also called PPARD) and PPARγ (also called PPARG). In special preferred embodiments, the treatment influences (such as increases or decreases, preferably increases) the expression of at least one receptor selected from the group of CNR1, CNR2, GPR84, GPR55 and PPARγ. In special preferred embodiments, the treatment influences (such as increases or decreases, preferably increases) the expression of CNR1, CNR2 and CPR84. In other special preferred embodiments, the treatment influences (such as increases or decreases, preferably increases) the expression of GPR55 and PPARG.

Preferably, the therapeutic composition against cancer comprising at least one cannabinoid, more preferably the at least one cannabinoid, interacts (such as associates to, binds to, agonizes and/or antagonizes) with at least one receptor selected from the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA), PPARβ (also called PPARD) and PPARγ (also called PPARG). Preferably, the treatment influences (such as increases or decreases) the expression of at least one secretory cytokine selected from the group consisting of EGF, FGF, VEGF, TNF, and Interleukins. In special preferred embodiments, the therapeutic composition against cancer comprising at least one cannabinoid, more preferably the at least one cannabinoid, interacts (such as associates to, binds to, agonizes and/or antagonizes) with at least one receptor selected from the group of CNR1, CNR2, GPR84, GPR55 and PPARγ. In special preferred embodiments, the therapeutic composition against cancer comprising at least one cannabinoid, more preferably the at least one cannabinoid, interacts (such as associates to, binds to, agonizes and/or antagonizes) with at least one receptor selected from the group of CNR1, CNR2 and CPR84. In other special preferred embodiments, the therapeutic composition against cancer comprising at least one cannabinoid, more preferably the at least one cannabinoid, interacts (such as associates to, binds to, agonizes and/or antagonizes) with at least one receptor selected from the group of GPR55 and PPARG.

Preferably, the treatment influences (such as increases or decreases) the expression of at least one secretory cytokine selected from the group consisting of EGF, FGF, VEGF, TNF, and Interleukins. In special preferred embodiments, the treatment influences (such as increases or decreases, preferably increases) the expression of at least one secretory cytokine selected from structural families of four-α-helix bundle (preferably selected from the group of IL-2 subfamily, IFN subfamily and IL-10 subfamily), IL-1, cysteine knot cytokines and IL-17 family. In special preferred embodiments, the treatment influences (such as increases or decreases, preferably increases) the expression of at least one secretory cytokine selected from the group of EGF, FGF, VEGF, IL-1α, IL-1β, IL-1RA, IL-18, Common g chain (CD132), IL-2, IL-4, IL-7, IL-9, IL-13, IL-15, Common b chain (CD131), IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP. In special preferred embodiments, the treatment influences (such as increases or decreases, preferably increases) the expression of at least one secretory cytokine selected from the group consisting of EGF, FGF, VEGF, TNF, IFN, TGF, TRAIL, SCF, MSP, CD, OSM, BBL, GITRI, LIGHT, OX, TALL, TWEAK, TRANCE and Interleukins. In special preferred embodiments, the treatment influences (such as increases or decreases, preferably increases) the expression of at least one secretory cytokine selected from the group consisting of EGF, FGF, VEGF, TNF and Interleukins.

Preferably, the composition comprising at least one cannabinoid, more preferably the at least one at least one cannabinoid, interacts with (for example binds to, deactivates, competes with) at least one secretory cytokine selected from the group consisting of EGF, FGF, VEGF, TNF, and Interleukins. In special preferred embodiments, the treatment interacts with (for example binds to, deactivates, competes with) at least one secretory cytokine selected from structural families of four-α-helix bundle (preferably selected from the group of IL-2 subfamily, IFN subfamily and IL-10 subfamily), IL-1, cysteine knot cytokines and IL-17 family. In special preferred embodiments, the treatment interacts with (for example binds to, deactivates, competes with) at least one secretory cytokine selected from the group of EGF, FGF, VEGF, IL-1α, IL-1β, IL-1RA, IL-18, Common g chain (CD132), IL-2, IL-4, IL-7, IL-9, IL-13, IL-15, Common b chain (CD131), IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, TGF-β1, TGF-β2, TGF-β3, Epo, Tpo, Flt-3L, SCF, M-CSF, MSP. In special preferred embodiments, the treatment interacts with (for example binds to, deactivates, competes with) at least one secretory cytokine selected at least one secretory cytokine selected from the group consisting of EGF, FGF, VEGF, TNF, IFN, TGF, TRAIL, SCF, MSP, CD, OSM, BBL, GITRI, LIGHT, OX, TALL, TWEAK, TRANCE, and Interleukins.

In preferred embodiments, the treatment influences (such as increases or decreases) the degree of phosphorylation of at least one intracellular signaling protein selected from the group consisting of MEK1, ERK1/2, AKT, FAK1, CHK2, JUN, CREB1, EGFR, RB, GSK3, HSP27, P38, IκB, NF-κB, mTOR, RSK1, SMAD3, AKT1S1, CHK2, MARCKS, p70S6K, LCK, PI3K, P53, PTN11, NRF2, STAT1, STAT3, STAT6, SRC and/or WNT. In special preferred embodiments, the treatment influences (such as increases or decreases, preferably increases) the degree of phosphorylation of at least one intracellular signaling protein selected from the group consisting of MEK1, ERK1/2, AKT, FAK1, CHK2, JUN, CREB1, EGFR, RB, GSK3, HSP27, P38, IκB, NF-κB, mTOR, RSK1, SMAD3, AKT1S1, CHK2, MARCKS, p70S6K, LCK, PI3K, P53, PTN11, NRF2, STAT1, STAT3, STAT6, SRC and/or WNT. In special preferred embodiments, the treatment influences (such as increases or decreases, preferably increases) the degree of phosphorylation of at least one intracellular signaling protein selected from the group consisting of MEK1/2, ERK1/2, P38, JUN, CREB, GSK3, STAT3, AKT, mTOR, AKT1S1, MARCKS, IKBA, SMAD3, HSP27 and P53.

In a fourth aspect, the invention pertains to an in vitro method for the personalization of cannabis-based cancer therapy, wherein the method comprises the steps:
(a) Providing a patient sample,
(b) Performing a multiomic analysis on said patient sample,
(c) Predicting the patient's response to a therapeutic composition comprising at least one cannabinoid, and
(d) Selecting a therapeutic composition comprising at least one cannabinoid for treatment of the patient based on the prediction of the patient's response.

In preferred embodiments of this aspect, the method comprises an additional step of cell testing as initial evaluation prior to performing step (b), preferably wherein the additional step of cell testing comprises administering to the patient sample a composition comprising at least one cannabinoid and determining a therapeutic effect. Preferably, the patient sample in this embodiment is isolated cancer cells.

In preferred embodiments of the invention, the method comprises an additional step of evaluating the efficiency of the therapeutic composition comprising at least one cannabinoid, preferably wherein the evaluating the efficiency of the therapeutic composition comprises administering to the patient sample a composition comprising at least one cannabinoid and determining a therapeutic effect. Preferably, the patient sample in this embodiment is isolated cancer cells.

In preferred embodiments of this aspect, the therapeutic composition comprising at least one cannabinoid is the therapeutic composition against cancer comprising at least one cannabinoid according to the other aspects of the invention.

In preferred embodiments of the invention the multiomic analysis of step (b) comprises a detection, optionally a quantification, of the biological marker of any one of the other aspects of the invention.

In preferred embodiments of the invention the multiomic analysis comprises the spatial single cell profiling, the gene expression analysis, the cytokine expression profiling and/or the protein phosphorylation analysis of any one of the other aspects of the invention.

In special preferred embodiments of the invention the multiomic analysis comprises the cell profiling, the gene expression analysis, the cytokine expression profiling and/or the protein phosphorylation analysis of any one of the other aspects of the invention.

In preferred embodiments of the invention, the patient sample is a solid tissue sample, a soft tissue sample or a bodily fluid. Preferably, the patient sample comprises a cell, preferably isolated cancer cells. The cell may be a healthy, diseased and/or a cancer cell. Preferably, the patient sample comprises a healthy cell.

In preferred embodiments of the invention, the patient sample comprises cancer cells. Preferably, this also includes patient samples comprising a single cancer cell.

In preferred embodiments of the invention. the predicting the patient's response to a therapeutic composition comprising at least one cannabinoid comprises comparing the data generated by step (b) to library data, preferably wherein the library data is the central database or the database of potential therapeutic cannabinoid targets.

In preferred embodiments of the invention, the library data comprises the predetermined chemical profile and the therapeutic efficacy of the composition comprising at least one cannabinoid according to the previous aspects of the invention.

In preferred embodiments of the invention, the patient's response to a composition comprising at least one cannabinoid further comprises using data from the computational analysis according to the other aspects of the invention, preferably the first aspect of the invention. In special preferred embodiments of the invention, the predicting a patient's response to a composition comprising at least one cannabinoid further comprises using data from the computational analysis according to the other aspects of the invention, preferably the first aspect of the invention.

In special preferred embodiments, the cancer, such as used in the expression “cannabis-based cancer therapy”, refers to a cancer according to any other aspect of the invention.

In a fifth aspect, the invention pertains to a method for identifying and/or optimizing a therapeutic composition against cancer comprising at least one cannabinoid, wherein the method comprises the steps:
(i) Manufacturing a composition comprising at least one cannabinoid with a predetermined chemical profile,
(ii) Determining the therapeutic efficacy of the composition comprising at least one cannabinoid.
(iii) Performing a computational analysis on the data obtained in step (i) or (ii).

“Optimizing” in this context refers to changing manufacturing parameters and/or chemical profile of the composition comprising at least one cannabinoid to increase its therapeutic effect. Preferably, this optimization is performed iteratively. The term “iteratively” indicates that the therapeutic effect of a first composition comprising at least one cannabinoid is determined. Subsequently, the therapeutic effect of a second composition comprising at least one cannabinoid is determined. If the therapeutic effect of the second composition comprising at least one cannabinoid is more favourable than the therapeutic effect of the first composition comprising at least one cannabinoid, then the second composition comprising at least one cannabinoid is selected. Therefore, the manufacturing parameters and/or the chemical profile of the second composition comprising at least one cannabinoid is selected.

Preferably, the computational analysis comprises correlating the predetermined chemical profile, with the therapeutic efficacy of the composition comprising at least one cannabinoid to create a central database.

Preferably, steps (ii) and (iii) of this fifth aspect are conducted analogously as the steps (i) and (ii) of the first aspect of the invention. Preferably, the determining the therapeutic efficacy of the composition comprising at least one cannabinoid is conducted according to the other aspects of the invention, preferably the first aspect. Preferably, the performing a computational analysis on the data obtained in step (i) or (ii) is performed according to the first aspect of the invention. In the context of this aspect, the data obtained in step (i) preferably is the predetermined chemical profile. In the context of this aspect, the data obtained in step (ii) is preferably the therapeutic efficacy of the composition comprising at least one cannabinoid. Preferably, the definitions of the other aspects of the inventions also apply to this aspect of the invention.

In special a sixth aspect, the invention pertains to a method for the treatment of a disease in a subject in need thereof, wherein the method comprises administering to the subject a therapeutic composition comprising at least one cannabinoid. In special preferred embodiments, the therapeutic composition comprising at least one cannabinoid is a therapeutic composition comprising at least one cannabinoid against cancer according to any other aspect of the invention. In special preferred embodiments, the disease is cancer.

In a special further aspect, the invention pertains to a method for identifying a therapeutic composition against cancer comprising at least one cannabinoid, wherein the method comprises the steps:
(i) Determining a therapeutic efficacy of the composition comprising at least one cannabinoid with a predetermined chemical profile,
(ii) Performing a computational analysis on the therapeutic efficacy of the composition comprising at least one cannabinoid with the predetermined chemical profile.

In yet a special further aspect, the invention pertains to a method for identifying and/or optimizing a therapeutic composition against cancer comprising at least one cannabinoid, wherein the method comprises the steps:
(i) Manufacturing a composition comprising at least one cannabinoid with a predetermined chemical profile,
(ii) Determining a therapeutic efficacy of the composition comprising at least one cannabinoid.
(iii) Performing a computational analysis on the data obtained in step (i) or (ii).

Moreover, the invention pertains to the following special itemized embodiments:

Item 1: A method for identifying a therapeutic composition against cancer comprising at least one cannabinoid, wherein the method comprises the steps:
(i) Determining the therapeutic efficacy of the composition comprising at least one cannabinoid with a predetermined chemical profile;
(ii) Performing a computational analysis on the therapeutic efficacy of the composition comprising at least one cannabinoid with the predetermined chemical profile.

Item 2: The method of item 1, wherein the determining a therapeutic efficacy comprises comparing a biological marker of a sample that is not exposed to the composition comprising at least one cannabinoid to the biological marker of sample that is exposed to the composition comprising at least one cannabinoid, wherein the increase and/or decrease of the biological marker is indicative of a therapeutic effect such as cancer cell death, impairment of tumor angiogenesis and/or inhibition of cancer cell proliferation, cancer cell adhesion, cancer cell migration, cancer cell invasiveness and/or other harmful effects of cancer cells, preferably, wherein the comparing a biological marker comprises conducting a multiomic analysis.

Item 3: The method of any one of items 1 or 2, wherein the determining the therapeutic efficacy comprises conducting an assay that is suitable for the detection of a cancer cell function, preferably wherein the cancer cell function is cell proliferation and/or cell death.

Item 4: The method of any one of items 1 to 3, wherein the multiomic analysis comprises
spatial single cell profiling of the sample, preferably using a multiplexed immunohistochemistry method, preferably wherein the multiplexed immunohistochemistry method comprises a detection, optionally a quantification, of a cannabinoid-related receptor, preferably, wherein the cannabinoid-related receptor is selected from at least one cannabinoid-related receptor of the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA) and PPARβ (also called PPARD), PPARγ (also called PPARG),
a cell composition analysis, a neighborhood analysis, a comprises and/or a, preferably spatial, gene expression analysis, preferably wherein the gene expression analysis comprises a transcriptome analysis and, optionally, a transcription factor analysis and/or a pathway analysis,
a cytokine expression profiling, preferably comprising a detection, optionally a quantification, of at least one secretory cytokine selected from the group consisting of EGF, FGF, VEGF, TNF and Interleukins, and/or
a protein phosphorylation analysis, preferably comprising a detection, optionally a quantification, of the phosphorylation of at least one intracellular signaling protein selected from the group consisting of MEK1, ERK1/2, AKT, FAK1, CHK2, JUN, CREB1, EGFR, RB, GSK3, HSP27, P38, IκB, NF-κB, mTOR, RSK1, SMAD3, AKT1S1, CHK2, MARCKS, p70S6K, LCK, PI3K, P53, PTN11, NRF2, STAT1, STAT3, STAT6, SRC and/or WNT.

Item 5: The method of any one of items 1 to 4, wherein the computational analysis comprises correlating the predetermined chemical profile with the therapeutic efficacy of the composition comprising at least one cannabinoid to create a central database.

Item 6: The method of any one of items 1 to 5, wherein the computational analysis comprises the use of an in silico model, preferably wherein the in silico model describes metabolic pathways and/or gene expression of a subject (such as of a subject suffering from cancer) or preferably of a cell (such as of a cancer cell), when exposing the subject or preferably the cell to the composition comprising at least one cannabinoid.

Item 7: The method of any one of items 1 to 6, wherein the computational analysis comprises:
creating and/or visualizing data networks, analyzing nodes, and/or performing cluster analyses;
the creation of a database of potential therapeutic cannabinoid targets, preferably wherein the database of potential therapeutic cannabinoid targets is created by the use of in silico target prediction methods, preferably wherein in silico target prediction methods are selected from SwissTarget, BioGRID, canCAR and/or the CaNDis web server; and/or
steps for identifying therapeutic cannabinoid targets comprising matching of a potential therapeutic cannabinoid target with its gene data, preferably by using the Enrichr toolset and/or matching a potential therapeutic cannabinoid target by its presence in key biological pathways, preferably by using the KEGG database;
preferably, wherein the potential therapeutic cannabinoid targets
are linked to common interaction partners in the protein-protein interaction String database, optionally wherein additional therapeutic targets and/or mechanisms of observed adverse drug effects are identified and/or
are in the form of experimental crystal complexes such as in the RCSB PDB Database and/or in the form of homology models such as in SwissModel, Modeller and/or Yasara.

Item 8: The method of item 7, further comprising studying the interaction of cannabinoids, preferably cannabinoids of the composition comprising at least one cannabinoid, and their metabolites with the potential therapeutic cannabinoid targets, preferably at an atomistic level, and/or
wherein the interaction of the cannabinoids and/or their metabolites with their potential therapeutic cannabinoid targets is evaluated by using direct methods, preferably inverse molecular docking, and/or by constructing drug-target interaction fingerprints, maps and/or structure-activity-relationships.

Item 9: The method according to any one of items 1 to 8, comprising setting up mechanistic models of action of individual cannabinoids and/or combinations of cannabinoids, preferably wherein the mechanistic models of action of individual cannabinoids and/or combinations of cannabinoids are suitable to rationally set effective therapy for selected cancer patients.

Item 10: A therapeutic composition against cancer comprising at least one cannabinoid, wherein the therapeutic composition against cancer comprising at least one cannabis extract is a therapeutic composition obtainable by the method according to items 1 to 8.

Item 11: The therapeutic composition against cancer comprising at least one cannabis extract of item 10, wherein the cannabinoid is a phytocannabinoid and/or comprises at least one pharmaceutically acceptable carrier and/or excipient.

Item 12: A therapeutic composition against cancer comprising at least one cannabinoid according to item 10 to 11 for use in the treatment of a condition or disease, preferably wherein the disease is cancer.

Item 13: An in vitro method for the personalization of cannabis-based cancer therapy, wherein the method comprises the steps:
(a) Providing a patient sample, preferably a solid tissue sample, a soft tissue sample or a bodily fluid, preferably wherein the patient sample comprises cancer cells,
(b) Performing a multiomic analysis on said patient sample, preferably wherein the multiomic analysis comprises a detection, optionally a quantification, of the biological marker of any one of items 2 to 4,
(c) Predicting the patient's response to a therapeutic composition comprising at least one cannabinoid, preferably, wherein the therapeutic composition comprising at least one cannabinoid is the therapeutic composition against cancer comprising at least one cannabinoid according to any one of items 10 to 12, and
(d) Selecting a therapeutic composition comprising at least one cannabinoid for treatment of the patient based on the prediction of the patient's response,
   wherein the method optionally comprises
   an additional step of cell testing as initial evaluation prior to performing step (b), preferably wherein the additional step of cell testing comprises administering to the patient sample a composition comprising at least one cannabinoid and determining a therapeutic effect according to item 2, and/or
   comprising an additional step of evaluating the efficiency of the therapeutic composition comprising at least one cannabinoid, preferably wherein the evaluating the efficiency of the therapeutic composition comprises administering to the patient sample a composition comprising at least one cannabinoid and determining a therapeutic effect according to item 2.

Item 14: The in vitro method of any one of items 13, wherein the predicting the patient's response to a composition comprising at least one cannabis extract comprises comparing the data generated by step (b) to library data, preferably wherein the library data is the central database of item 5 or the database of potential therapeutic cannabinoid targets according to item 7, preferably wherein the library data comprises the predetermined chemical profile and the therapeutic efficacy of the composition comprising at least one cannabinoid according to the method of any one of the items 1 to 9 and/or wherein the predicting the patient's response to a composition comprising at least one cannabinoid further comprises using data from the computational analysis according to the method of items 1 to 9.

Item 15: A method for identifying and/or optimizing a therapeutic composition against cancer comprising at least one cannabinoid, wherein the method comprises the steps:
(i) Manufacturing a composition comprising at least one cannabinoid with a predetermined chemical profile,
(ii) Determining the therapeutic efficacy of the composition comprising at least one cannabinoid according to any one of items 1 to 9.
(iiI) Performing a computational analysis on the data obtained in step (i) or (ii), wherein the computational analysis is the computational analysis according to any one of items 5 to 9.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES

The figures show:

FIG. 1: Demonstrates a heatmap of average docking results for identified protein classes from human proteome. Black color indicates no binding while bright grey designates potentially favorable binding interaction.

FIG. 2: Depicts a focused heatmap where the 10 best scoring receptors of FIG. 3 are pooled from each individual cannabinoid and classified. While the data shown is based on the 10 best scoring receptors for the individual cannabinoids, the heatmap also shows the binding interaction of the individual cannabinoids with the best scoring targets of the other cannabinoids. One cell represents one cannabinoid docking score on one target, for example, the 29 datapoints of hydrolases represent 29 different hydrolases and their scores against the cannabinoid compounds. Black color indicates no binding while bright grey designates potentially favorable binding interaction.

FIG. 3: Heatmap of docking results of the 100 receptors with the best average docking scores. Further details on the receptors can be found in Table 2. In short, the first four letters of the receptor codes refer to RCSB PDB database 4 letter codes (https://www.rcsb.org/, dated 27 Jun. 2023), the last letter of the receptor code refers to the chain of the protein that was used as a receptor. Black color indicates no binding while bright grey designates potentially favorable binding interaction.

FIG. 4: (A)-(P) Presentation of distinct docking score distribution graphs of the cannabinoid ligands on the human proteome.

FIG. 5: Heatmap of the average inverse docking results for focused inverse docking library of assay intracellular proteins for in vitro biological evaluation. Black color indicates no binding while bright grey designates potentially favorable binding interaction.

FIG. 6: Heatmap of the average inverse docking results for classified focused inverse docking library of assay intracellular proteins for in vitro biological evaluation. Black color indicates no binding while bright grey designates potentially favorable binding interaction.

FIG. 7: Heatmap of the average inverse docking results for classified focused inverse docking library of assay receptor proteins for in vitro biological evaluation. The figure shows inverse docking fingerprints for 16 cannabinoid compounds on 21 postulated receptors (CNR1, CNR2, TRPV1, TRPV2, TRPV3, TRPV4, TRPA1, TRPM8, GPR55, GPR35, GPR119, GPR18, GPR12, GPR84, GPR3, GPR6, LPAR4, LPAR5, PPARA, PPARD, PPARG). Black color indicates no binding while bright grey designates potentially favorable binding interaction.

FIG. 8: Basal mRNA expression of CNR1, CNR2, GPR84, GPR55 and PPARγ in melanoma cell lines.

FIG. 9: Basal mRNA expression of CNR1, CNR2, GPR84, GPR55 and PPARγ in colon cancer cell lines using real-time quantitative PCR.

FIG. 10: shows cell viability assessment for 16 cannabinoid compounds on 2 colon cancer cell lines (HCT116 and CaCO2. (A) shows dose response curves for each cannabinoid as % relative to untreated control. Part (B) shows the area under the curve where dark color indicates stronger decrease in cell viability and bright color weaker decrease in viability.

FIG. 11: Shows phosphoprotein expression profiles of CaCo2 and HCT116 colon cancer cells treated with 5 cannabinoids (CBN, CBG, CBD, CBC, THC). Results are represented as a change to the control on a log-scale.

FIG. 12: Shows a model of signaling network of the endocannabinoid system in cancer.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

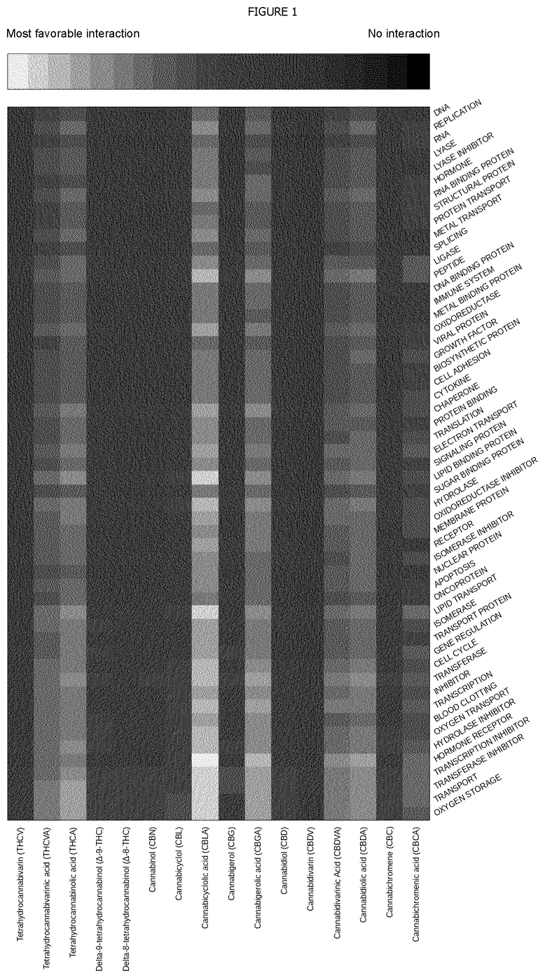

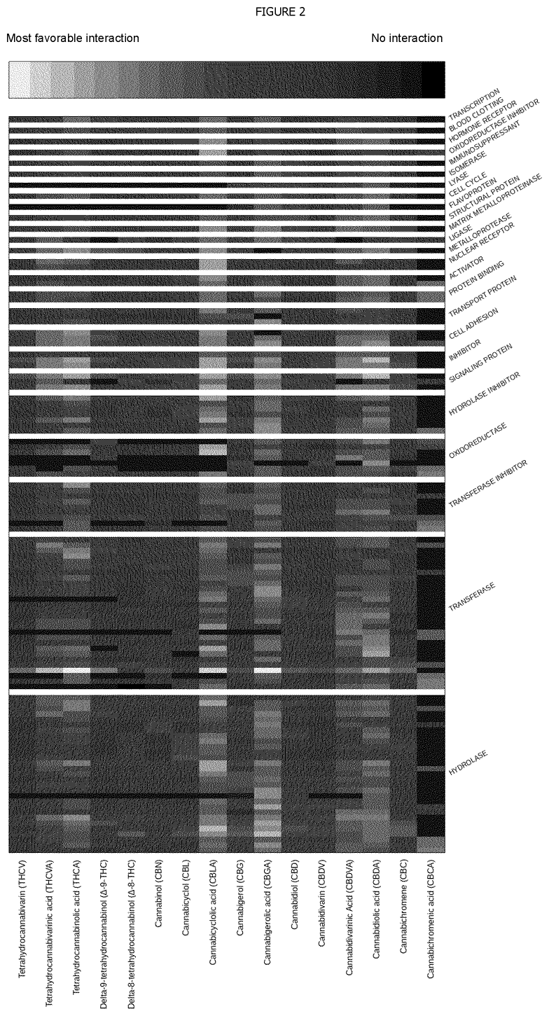

Example 1: Inventors performed and inverse docking study on a focused library of 16 cannabinoid compounds (Δ9-THCV-C3, Δ9-THCVA-C3 A, Δ9-THCA-C5 A, Δ9-THC-C5, Δ8-THC-C5, CBN-C5, CBL-C5, CBLA-C5 A, (E)-CBG-C5, (E)-CBGA-C5 A, CBD-C5, CBDV-C3, CBDVA-C3, CBDA-C5, CBC-C5, and CBCA-C5 A) on a complete human proteome to identify a very wide targeting space for cannabinoid compounds encompassing targets from multiple classes such as: hydrolases, transferases, oxidoreductases, signaling proteins, cell adhesion proteins, transport proteins, nuclear receptors, metalloproteases, ligases, matrix metalloproteases, flavoproteins, cell cycle related proteins, lyases, isomerases, proteins involved in immunological response, hormone receptor, proteins involved in blood, transcription and apoptotic proteins. The potential target space is much wider than previously acknowledged in literature and ideal for detail cannabinoid or composition studies, stratification and biological pathway targeting strategy definition. The identified protein classes along with average inverse docking scores for studied cannabinoids are represented in FIG. 1. Furthermore FIG. 2 represents a focused heatmap where inventors pooled together the 10 best scoring receptors from each individual ligand. Identified sorted pools of classified receptors indicate a wide potential biological target involvement of cannabinoid compounds.

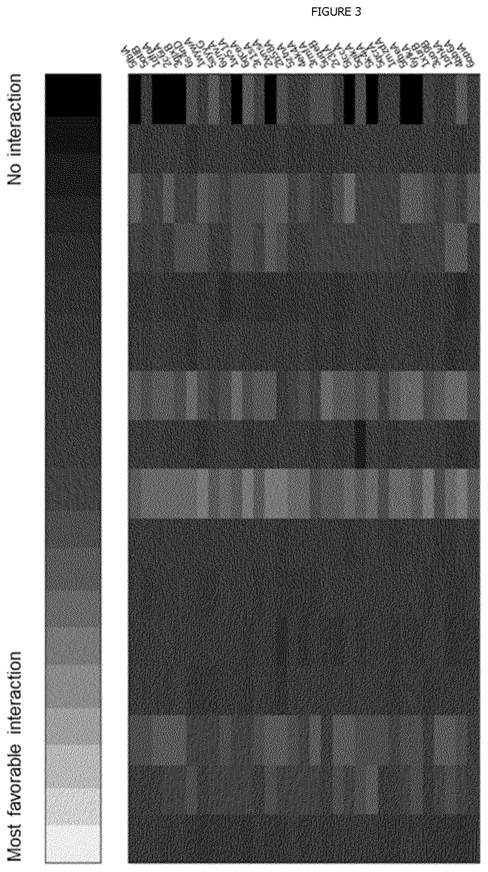

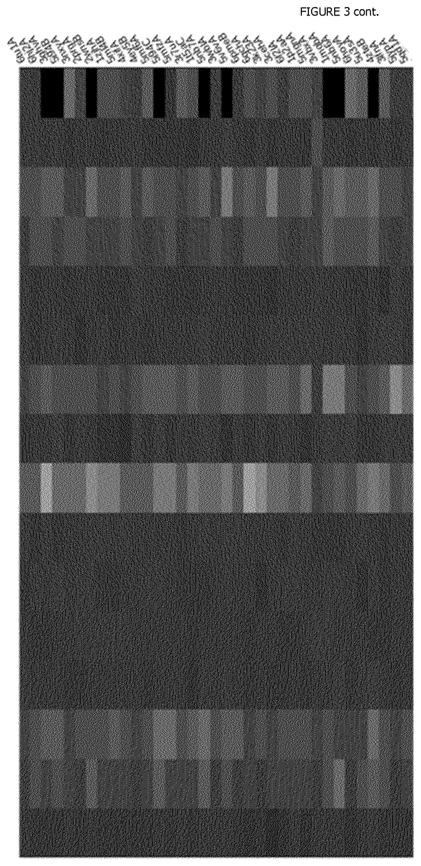

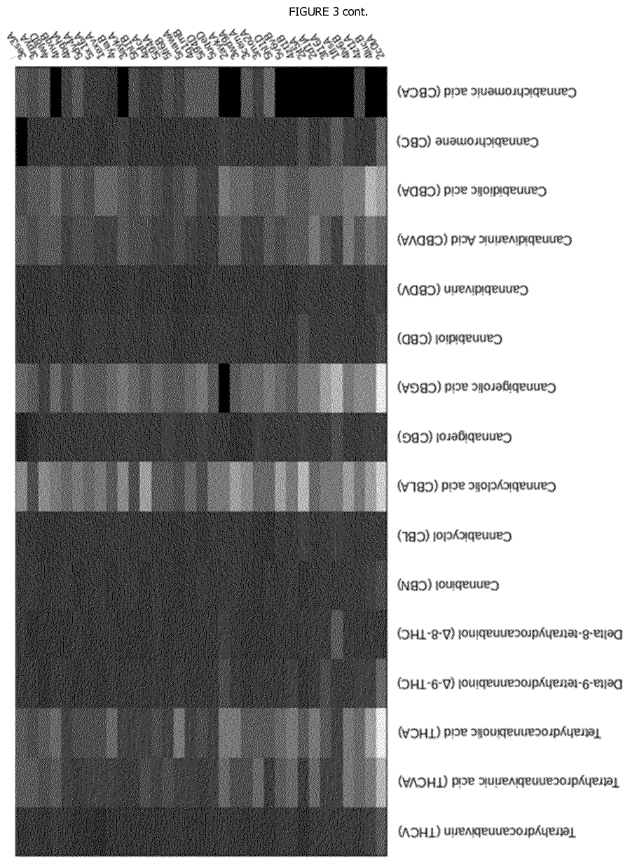

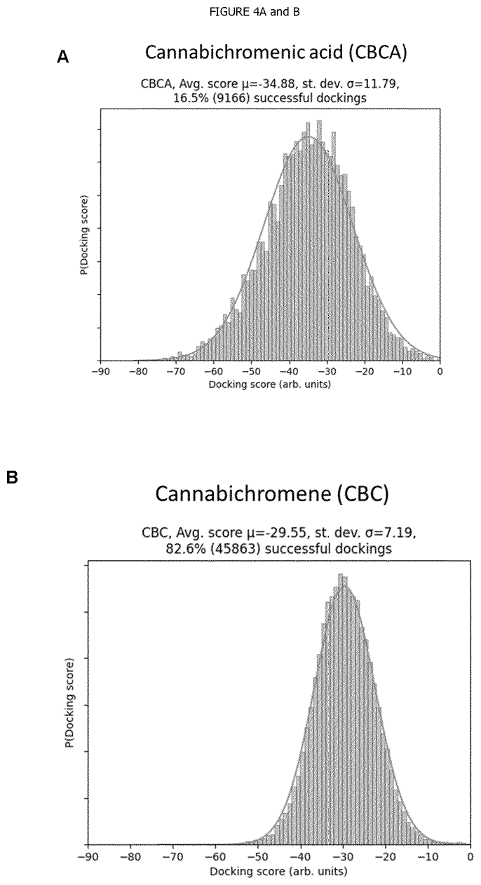

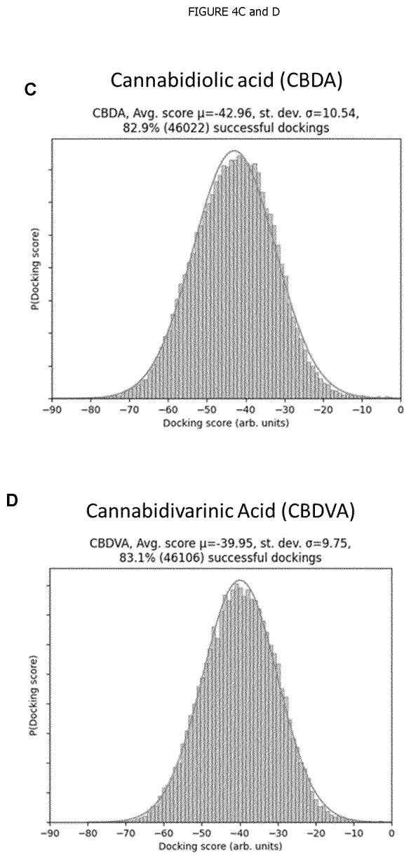

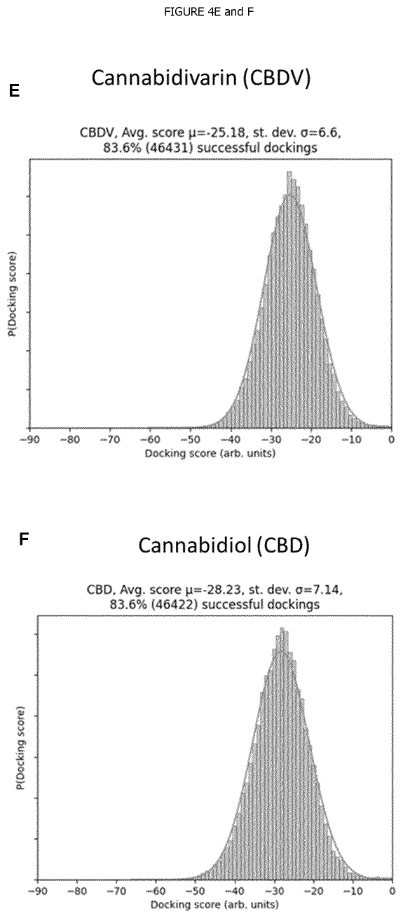

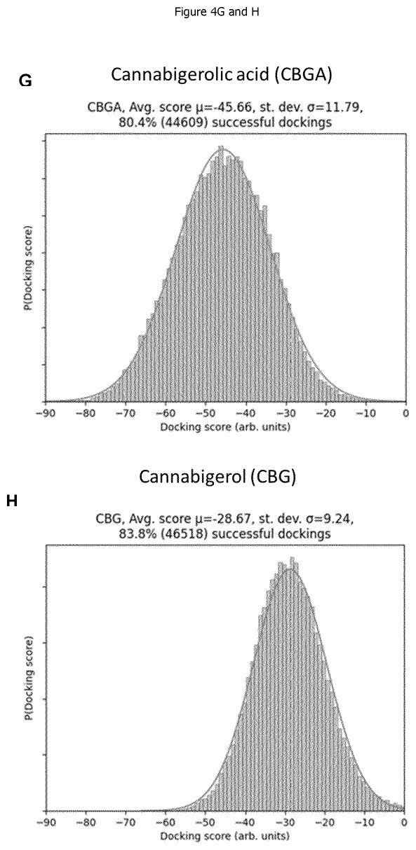

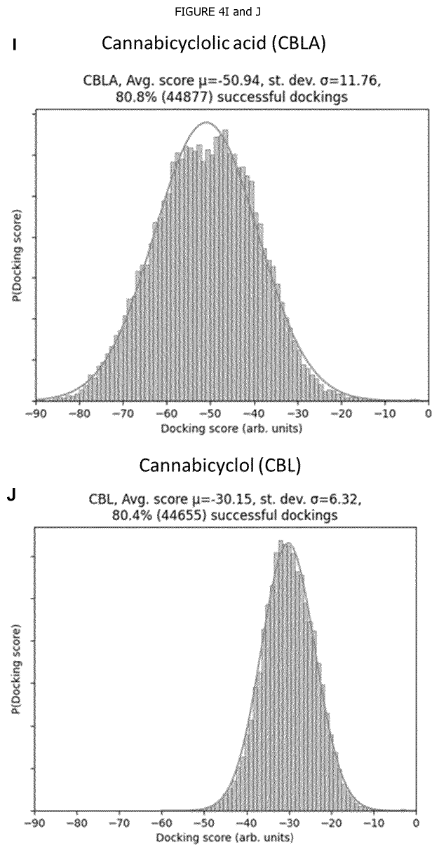

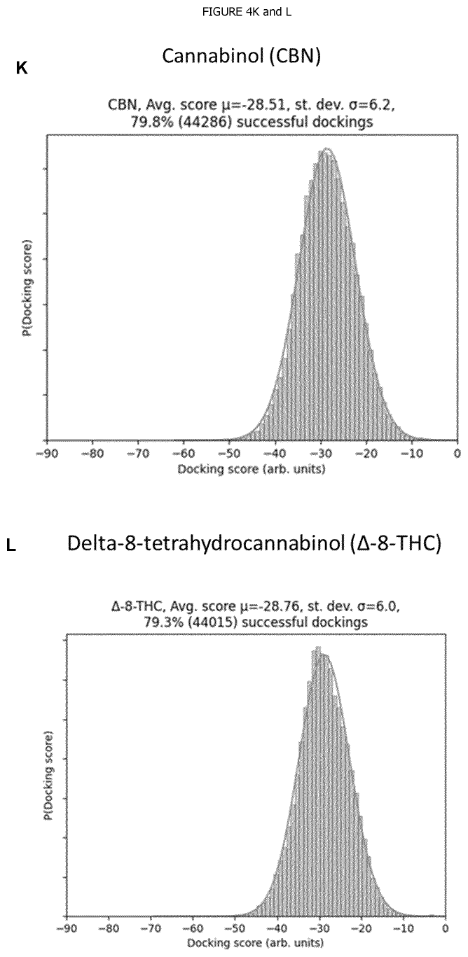

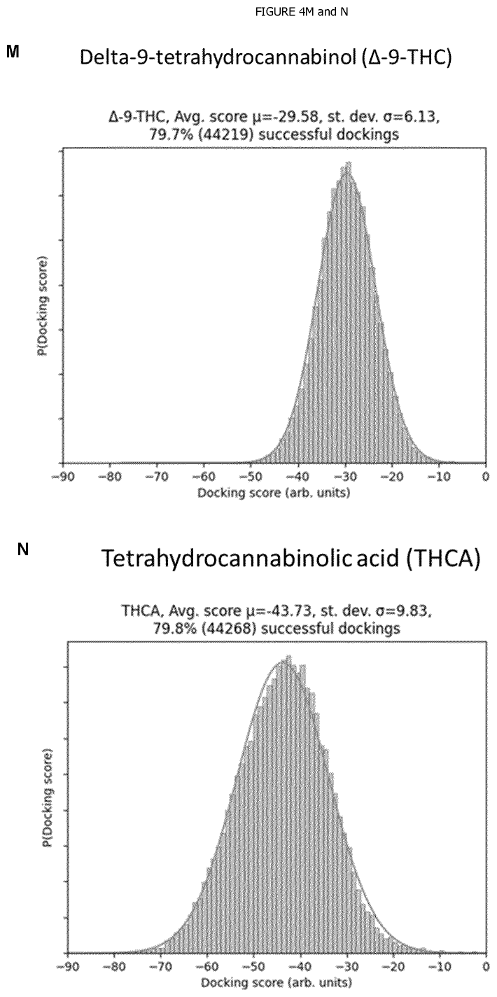

Example 2: Inventors performed a structural study on a focused library of 16 cannabinoid compounds (Δ9-THCV-C3, Δ9-THCVA-C3 A, Δ9-THCA-C5 A, Δ9-THC-C5, Δ8-THC-C5, CBN-C5, CBL-C5, CBLA-C5 A, (E)-CBG-C5, (E)-CBGA-C5 A, CBD-C5, CBDV-C3, CBDVA-C3, CBDA-C5, CBC-C5, and CBCA-C5 A). Cannabinoids share a common secondary metabolite class but nevertheless display unique chemical structures and have distinct pharmacophoric profiles. Namely, the inventors performed a molecular selectivity study on a complete database of proteins (namely the RCSB PDB Database; https://www.rcsb.org/) from the human genome (55508 receptor structures). The inventors used the ProBiS-Dock docking algorithm and reconstructed cannabinoid binding to the receptor from small cannabinoid fragments. Further information on the ProBiS-Dock docking algorithm can be found in Konc et al (16). Workflow outputted multiple binding poses an/or possibility of binding of cannabinoids to human proteins along with assessment of relative free binding energy via ProBiS-Dock docking scores. FIG. 3 displays results of the 100 receptors with the best docking scores on average where distinct differences in overall binding to the complete set of 100 receptors can be observed. Namely THCA, THCVA, CBLA, CBGA, CBDVA, CBDA display a markedly higher docking scores and distinct binding profiles when compared to the rest of the library set or CBCA that represents the low binding spectrum cannabinoid and again, distinct binding profile. The top scoring receptors (see Table 2) are identified as: 2c0tA, 4lucB, 4zt1A, 4lv6B, 1flsA, 3f16A, 2d1jA, 2jt5A, 4zt1B, 5v6vB, 5hl1D, 3mo2A, 3cemA, 3wd9A, 2aykA, 5uqeD, 5i94D, 4g1mB, 5nawA, 5fi6B, 5i94A, 4qfcA, 5hl1B, 3aykA, 4yiaB, 1exvA, 5x16A, 5dy4A, 4bghA, 4nvqB, 4wj8D, 3rpyA, 3es3A, 5qd1A, 5jypA, 3lfnA, 4zteB, 5u3sA, 6hoyA, 5nb6A, 1mqbA, 3vbxA, 5mgnA, 1p2aA, 6f2lA, 3cehA, 3k23A, 6qchA, 6pmeB, 5v6vA, 3wblA, 5nb7A, 1t5aC, 3r7uA, 5mfzA, 5i94C, 5mf6A, 4ey5B, 4xifA, 5ml4B, 1zjhA, 2wnsB, 2prgA, 3nxyA, 5i94B, 3ghvA, 6hj2A, 6tu1A, 6opiA, 4bb6A, 1ohkA, 3uo9B, 1xurB, 6ylkA, 5fbeA, 1mzdA, 5qcrA, 5k4jA, 5qikA, 5tccA, 2r3jA, 5uqeB, 3rmfA, 4ek4A, 5ztnA, 2b58A, 2wnsA, 3r1sA, 6qceA, 1w51A, 6vnvA, 5ayyG, 1wywA, 6s4nD, 3gjxB, 2c6iA, 1dfpA, 50tlB, 5fbiA, 4hdaA. Distinct pharmacophoric profiles can be observed from average docking scores of the cannabinoids from the focused library on the examined target space as is displayed in FIG. 4A-P.

TABLE 2
PDB ID and CHAIN of the top scoring receptors, as well as their average docking scores and classification.

| | Average | | |
|---|---|---|---|
| PDB ID | docking | | |
| and CHAIN | score | Classification | Descriptor |
| 2c0tA | −71.55 | TRANSFERASE | TYROSINE-PROTEIN KINASE HCK (E.C.2.7.1.112) |
| 4lucB | −63.96 | SIGNALING | GTPase KRas |
| | | PROTEIN/INHIBITOR | |
| 4zt1A | −61.07 | CELL ADHESION | Cadherin-3 |
| 4lv6B | −60.82 | SIGNALING | GTPase KRas |
| | | PROTEIN/INHIBITOR | |
| 1flsA | −59.48 | HYDROLASE | SOLUTION STRUCTURE OF THE CATALYTIC FRAGMENT OF |
| | | | HUMAN COLLAGENASE-3 (MMP-13) COMPLEXED WITH A |
| | | | HYDROXAMIC ACID INHIBITOR |
| 3f16A | −57.58 | HYDROLASE | Crystal structure of the catalytic domain of human MMP12 |
| | | | complexed with the inhibitor (R)-N-(3-hydroxy-1-nitroso-1- |
| | | | oxopropan-2-yl)-4-methoxybenzenesulfonamide |
| 2d1jA | −57.57 | HYDROLASE | Coagulation factor X, heavy chain (E.C.3.4.21.6), |
| | | | Coagulation factor X, light chain (E.C.3.4.21.6) |
| 2jt5A | −55.38 | HYDROLASE | Stromelysin-1 (E.C.3.4.24.17) |
| 4zt1B | −55.08 | CELL ADHESION | Cadherin-3 |
| 5v6vB | −55.02 | HYDROLASE/HYDROLASE | GTPase KRas |
| | | INHIBITOR | |
| 5hl1D | −54.98 | HYDROLASE/HYDROLASE | Glutaminase kidney isoform, mitochondrial (E.C.3.5.1.2) |
| | | INHIBITOR | |
| 3mo2A | −54.74 | TRANSFERASE | Histone-lysine N-methyltransferase, H3 lysine-9 specific 5 |
| | | | (E.C.2.1.1.43) |
| 3cemA | −54.1 | TRANSFERASE | Glycogen phosphorylase (E.C.2.4.1.1) |
| 3wd9A | −53.86 | HYDROLASE/HYDROLASE | cAMP-specific 3′,5′-cyclic phosphodiesterase 4B |
| | | INHIBITOR | (E.C.3.1.4.17) |
| 2aykA | −53.6 | METALLOPROTEASE | INHIBITOR-FREE CATALYTIC FRAGMENT OF HUMAN |
| | | | FIBROBLAST COLLAGENASE, NMR, MINIMIZED AVERAGE |
| | | | STRUCTURE |
| 5uqeD | −53.44 | HYDROLASE | Glutaminase liver isoform, mitochondrial (E.C.3.5.1.2) |
| 5i94D | −52.71 | HYDROLASE/HYDROLASE | Glutaminase kidney isoform, mitochondrial (E.C.3.5.1.2) |
| | | INHIBITOR | |
| 4g1mB | −52.55 | PROTEIN BINDING | Integrin alpha-V, Integrin beta-3 |
| 5nawA | −52.35 | HYDROLASE | Complement factor D (E.C.3.4.21.46) |
| 5fi6B | −52.3 | HYDROLASE/HYDROLASE | human glutaminase C |
| | | INHIBITOR | |
| 5i94A | −52.22 | HYDROLASE/HYDROLASE | Glutaminase kidney isoform, mitochondrial (E.C.3.5.1.2) |
| | | INHIBITOR | |
| 4qfcA | −51.96 | OXIDOREDUCTASE/OXI- | D-amino-acid oxidase (E.C.1.4.3.3) |
| | | DOREDUCTASE INHIBITOR | |
| 5hl1B | −51.94 | HYDROLASE/HYDROLASE | Glutaminase kidney isoform, mitochondrial (E.C.3.5.1.2) |
| | | INHIBITOR | |
| 3aykA | −51.86 | MATRIX | COLLAGENASE (E.C.3.4.24.7) |
| | | METALLOPROTEINASE | |
| 4yiaB | −51.81 | SIGNALING PROTEIN | Thyroxine binding globulin |
| 1exvA | −51.73 | TRANSFERASE | GLYCOGEN PHOSPHORYLASE, PYRIDOXAL-5′-PHOSPHATE, |
| | | | 1-{2-[5-CHLORO-1H-INDOLE-2-CARBONYL)-AMINO]-3- |
| | | | PHEYNYL-PROPIONYL}-AZETIDINE-3-CARBOXYLIC ACID |
| 5x16A | −51.72 | HYDROLASE | NAD-dependent protein deacetylase sirtuin-6 (E.C.3.5.1.—) |
| 5dy4A | −51.66 | HYDROLASE | NAD-dependent protein deacetylase sirtuin-2 (E.C.3.5.1.—) |
| 4bghA | −51.66 | TRANSFERASE | CYCLIN-DEPENDENT KINASE 2 (E.C.2.7.11.22) |
| 4nvqB | −51.58 | TRANSFERASE/TRANSFERASE | Histone-lysine N-methyltransferase EHMT2 (E.C.2.1.1.—, |
| | | INHIBITOR | 2.1.1.43) |
| 4wj8D | −51.5 | TRANSFERASE | Pyruvate kinase isozymes M1/M2 (E.C.2.7.1.40) |
| 3rpyA | −51.46 | TRANSFERASE/TRANSFERASE | Cyclin-dependent kinase 2 (E.C.2.7.11.22) |
| | | INHIBITOR | |
| 3es3A | −51.42 | OXIDOREDUCTASE | Directing Noble Metal Ion Chemistry within a Designed |
| | | | Ferritin Protein. The Complex with Gold ions. |
| | | | Ferritin H8-H9x Mutant |
| 5qd1A | −51.38 | HYDROLASE | Beta-secretase 1 (E.C.3.4.23.46) |
| 5jypA | −51.25 | HYDROLASE | Glutaminase kidney isoform, mitochondrial (E.C.3.5.1.2) |
| 3lfnA | −51.22 | TRANSFERASE | Cell division protein kinase 2 (E.C.2.7.11.22) |
| 4zteB | −51.17 | CELL ADHESION | Cadherin-3 |
| 5u3sA | −51.17 | PROTEIN | Peroxisome proliferator-activated receptor delta |
| | | BINDING/ACTIVATOR | |
| 6hoyA | −51.11 | HYDROLASE | NAD-dependent protein deacetylase sirtuin-6 (E.C.3.5.1.—) |
| 5nb6A | −51.04 | HYDROLASE | Complement factor D (E.C.3.4.21.46) |
| 1mqbA | −51.02 | TRANSFERASE | Ephrin type-A receptor 2 (E.C.2.7.1.112) |
| 3vbxA | −50.95 | TRANSFERASE/TRANSFERASE | Serine/threonine-protein kinase pim-1 (E.C.2.7.11.1) |
| | | INHIBITOR | |
| 5mgnA | −50.94 | HYDROLASE | Human SIRT6 (E.C.3.5.1.17) |
| 1p2aA | −50.87 | TRANSFERASE | Cell division protein kinase 2, 5-[(2-AMINOETHYL)AMINO]- |
| | | | 6-FLUORO-3-(1H-PYRROL-2-YL)BENZO[CD]INDOL-2(1H)-ONE |
| 6f2lA | −50.77 | TRANSCRIPTION | Peroxisome proliferator-activated receptor gamma |
| 3cehA | −50.74 | TRANSFERASE | Glycogen phosphorylase (E.C.2.4.1.1) |
| 3k23A | −50.72 | TRANSCRIPTION | Glucocorticoid Receptor with Bound D-prolinamide 11 |
| 6qchA | −50.65 | HYDROLASE | NAD-dependent protein deacetylase sirtuin-6 (E.C.3.5.1.—) |
| 6pmeB | −50.63 | TRANSFERASE | High affinity nerve growth factor receptor (E.C.2.7.10.1) |
| 5v6vA | −50.6 | HYDROLASE/HYDROLASE | GTPase KRas |
| | | INHIBITOR | |
| 3wblA | −50.51 | TRANSFERASE/TRANSFERASE | Cyclin-dependent kinase 2 (E.C.2.7.11.22) |
| | | INHIBITOR | |
| 5nb7A | −50.51 | HYDROLASE | Complement factor D (E.C.3.4.21.46) |
| 1t5aC | −50.4 | TRANSFERASE | Pyruvate kinase, M2 isozyme (E.C.2.7.1.40) |
| 3r7uA | −50.29 | TRANSFERASE/TRANSFERASE | Cyclin-dependent kinase 2 (E.C.2.7.11.22) |
| | | INHIBITOR | |
| 5mfzA | −50.26 | HYDROLASE | Human SIRT6 (E.C.3.5.1.17) |
| 5i94C | −50.18 | HYDROLASE/HYDROLASE | Glutaminase kidney isoform, mitochondrial (E.C.3.5.1.2) |
| | | INHIBITOR | |
| 5mf6A | −50.13 | HYDROLASE | Human SIRT6 (E.C.3.5.1.17) |
| 4ey5B | −50.09 | HYDROLASE/HYDROLASE | Acetylcholinesterase (E.C.3.1.1.7) |
| | | INHIBITOR | |
| 4xifA | −50.09 | TRANSFERASE | Human O-GlcNAc transferase (E.C.2.4.1.255), VAL-THR- |
| | | | PRO-VAL-SER-THR-ALA-ALA |
| 5ml4B | −50.08 | LIPID BINDING PROTEIN | Retinal rod rhodopsin-sensitive cGMP 3′,5′-cyclic |
| | | | phosphodiesterase subunit delta |
| 1zjhA | −50.07 | TRANSFERASE | Pyruvate kinase, isozymes M1/M2 (E.C.2.7.1.40) |
| 2wnsB | −50.05 | TRANSFERASE | OROTATE PHOSPHORIBOSYLTRANSFERASE (E.C.2.4.2.10) |
| 2prgA | −50.02 | COMPLEX | PEROXISOME PROLIFERATOR ACTIVATED RECEPTOR |
| | | (THIAZOLIDINEDIONE/ | GAMMA, NUCLEAR RECEPTOR COACTIVATOR SRC-1, 2,4- |
| | | RECEPTOR) | THIAZOLIDIINEDIONE, 5-[[4-[2-(METHYL-2- |
| | | | PYRIDINYLAMINO)ETHOXY]PHENYL]METHYL]-(9CL) |
| 3nxyA | −50.01 | OXIDOREDUCTASE | Dihydrofolate reductase (E.C.1.5.1.3) |
| 5i94B | −49.97 | HYDROLASE/HYDROLASE | Glutaminase kidney isoform, mitochondrial (E.C.3.5.1.2) |
| | | INHIBITOR | |
| 3ghvA | −49.97 | OXIDOREDUCTASE | Human dihydrofolate reductase Q35K/N64F double mutant |
| | | | inhibitor complex |
| 6hj2A | −49.93 | NUCLEAR PROTEIN | Crystal structure of hPXR in complex with dabrafenib |
| 6tu1A | −49.84 | TRANSFERASE | CREB-binding protein (E.C.2.3.1.48) |
| 6opiA | −49.75 | TRANSFERASE | Mitogen-activated protein kinase 1 (E.C.2.7.11.24) |
| 4bb6A | −49.72 | OXIDOREDUCTASE | CORTICOSTEROID 11-BETA-DEHYDROGENASE ISOZYME 1 |
| | | | (E.C.1.1.1.146) |
| 1ohkA | −49.71 | OXIDOREDUCTASE | DIHYDROFOLATE REDUCTASE, NADPH DIHYDRO- |
| | | | NICOTINAMIDE-ADENINE-DINUCLEOTIDE PHOSPHATE, N- |
| | | | (4-CARBOXY-4-{4-[(2,4-DIAMINO-PTERIDIN-6-YLMETHYL)- |
| | | | AMINO]-BENZOYLAMINO}-BUTYL)-PHTHALAMIC ACID |
| 3uo9B | −49.7 | HYDROLASE/HYDROLASE | Glutaminase kidney isoform, mitochondrial (E.C.3.5.1.2) |
| | | INHIBITOR | |
| 1xurB | −49.68 | HYDROLASE | Collagenase 3 (E.C.3.4.24.—) |
| 6ylkA | −49.66 | TRANSFERASE | Cyclin-dependent kinase 2 (E.C.2.7.11.22) |
| 5fbeA | −49.64 | HYDROLASE | Kallikrein-7 (E.C.3.4.21.117) |
| 1mzdA | −49.6 | HYDROLASE | crystal structure of human pro-granzyme K |
| 5qcrA | −49.58 | HYDROLASE | Beta-secretase 1 (E.C.3.4.23.46) |
| 5k4jA | −49.55 | TRANSFERASE/TRANSFERASE | Cyclin-dependent kinase 2 (E.C.2.7.11.22) |
| | | INHIBITOR | |
| 5qikA | −49.48 | TRANSFERASE/TRANSFERASE | TGF-beta receptor type-1 (E.C.2.7.11.30) |
| | | INHIBITOR | |
| 5tccA | −49.48 | HYDROLASE/HYDROLASE | Complement factor D (E.C.3.4.21.46) |
| | | INHIBITOR | |
| 2r3jA | −49.42 | TRANSFERASE | Cell division protein kinase 2 (E.C.2.7.11.22) |
| 5uqeB | −49.42 | HYDROLASE | Glutaminase liver isoform, mitochondrial (E.C.3.5.1.2) |
| 3rmfA | −49.41 | TRANSFERASE/TRANSFERASE | Cyclin-dependent kinase 2 (E.C.2.7.11.22) |
| | | INHIBITOR | |
| 4ek4A | 49.39 | TRANSFERASE/TRANSFERASE | Cyclin-dependent kinase 2 (E.C.2.7.11.22) |
| | | INHIBITOR | |
| 5ztnA | −49.36 | TRANSFERASE/TRANSFERASE | Dual specificity tyrosine-phosphorylation-regulated kinase 2 |
| | | INHIBITOR | (E.C.2.7.12.1) |
| 2b58A | −49.3 | TRANSFERASE | Diamine acetyltransferase 1 (E.C.2.3.1.57) |
| 2wnsA | −49.29 | TRANSFERASE | OROTATE PHOSPHORIBOSYLTRANSFERASE (E.C.2.4.2.10) |
| 3r1sA | −49.26 | TRANSFERASE/TRANSFERASE | Cyclin-dependent kinase 2 (E.C.2.7.11.22) |
| | | INHIBITOR | |
| 6qceA | −49.24 | HYDROLASE | NAD-dependent protein deacetylase sirtuin-6 (E.C.3.5.1.—) |
| 1w51A | −49.24 | HYDROLASE/INHIBITOR | BETA-SECRETASE 1 (E.C.3.4.23.46) |
| 6vnvA | −49.23 | TRANSFERASE/INHIBITOR | Non-receptor tyrosine-protein kinase TYK2 (E.C.2.7.10.2) |
| 5ayyG | −49.23 | TRANSFERASE | NICOTINATE-NUCLEOTIDE PYROPHOSPHORYLASE |
| | | | [CARBOXYLATING] (E.C.2.4.2.19) |
| 1wywA | −49.22 | HYDROLASE | G/T mismatch-specific thymine DNA glycosylase (E.C.3.2.2.—), |
| | | | Ubiquitin-like protein SMT3C |
| 6s4nD | −49.21 | NUCLEAR PROTEIN | Oxysterols receptor LXR-beta |
| 3gjxB | −49.21 | PROTEIN TRANSPORT | Crystal Structure of the Nuclear Export Complex CRM1- |
| | | | Snurportin1-RanGTP |
| 2c6iA | −49.2 | TRANSFERASE | CELL DIVISION PROTEIN KINASE 2 (E.C.2.7.1.37) |
| 1dfpA | −49.17 | SERINE PROTEASE | FACTOR D, DIISOPROPYLPHOSPHONO GROUP |
| 5otlB | −49.14 | TRANSFERASE | Casein kinase II subunit alpha (E.C.2.7.11.1) |
| 5fbiA | −49.13 | HYDROLASE | Kallikrein-7 (E.C.3.4.21.117) |
| 4hdaA | −49.12 | HYDROLASE/HYDROLASE | NAD-dependent protein deacylase sirtuin-5, mitochondrial |
| | | ACTIVATOR | (E.C.3.5.1.—), Fluor-de-Lys peptide |

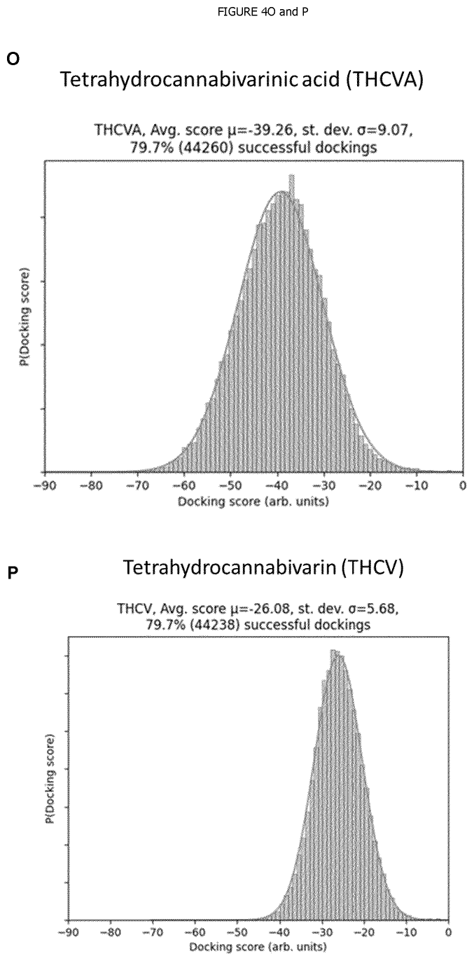

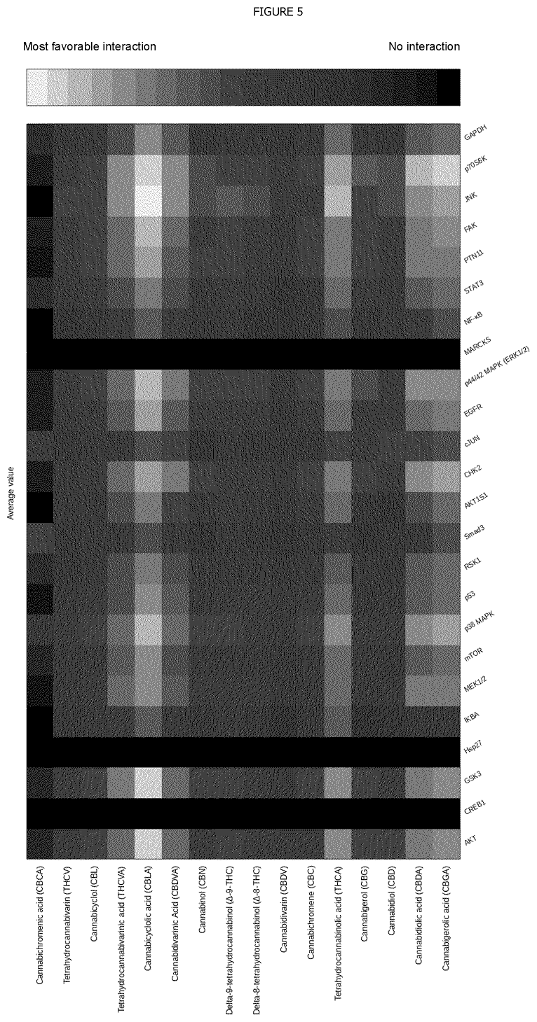

Example 3: The inventors performed focused inverse docking experiments on a library of 16 cannabinoid compounds (Δ9-THCV-C3, Δ9-THCVA-C3 A, Δ9-THCA-C5 A, Δ9-THC-C5, Δ8-THC-C5, CBN-C5, CBL-C5, CBLA-C5 A, (E)-CBG-C5, (E)-CBGA-C5 A, CBD-C5, CBDV-C3, CBDVA-C3, CBDA-C5, CBC-C5, and CBCA-C5 A). The inventors also employed a focused protein target library used for expression and signaling in vitro evaluation consisting of: AKT—RAC-alpha serine/threonine-protein kinase, CREB1—Cyclic AMP-responsive element-binding protein 1, GSK3—Glycogen synthase kinase-3 alpha, Hsp27—Heat shock protein beta-1, IKBA—NF-kappa-B inhibitor alpha, MEK1/2—Dual specificity mitogen-activated protein kinase kinase 1/Dual specificity mitogen-activated protein kinase kinase 2, mTOR—Serine/threonine-protein kinase mTOR, p38 MAPK—Mitogen-activated protein kinase 14/Mitogen-activated protein kinase 11/Mitogen-activated protein kinase 12, p53—Cellular tumor antigen p53, RSK1—Ribosomal protein S6 kinase alpha-1, Smad3—Mothers against decapentaplegic homolog 3, AKT1S1—Proline-rich AKT1 substrate 1, CHK2—Serine/threonine-protein kinase Chk2, cJUN—Transcription factor AP-1, EGFR—Epidermal growth factor receptor, p44/42 MAPK (ERK1/2) Mitogen-activated protein kinase 3/Mitogen-activated protein kinase 1, MARCKS—Myristoylated alanine-rich C-kinase substrate, NF-κB—Nuclear factor NF-kappa-B p105(100) subunit, STAT3—Signal transducer and activator of transcription 3, PTN11—Tyrosine-protein phosphatase non-receptor type 11, FAK—Focal adhesion kinase 1, JNK—Mitogen-activated protein kinase 9, p70S6K—Ribosomal protein S6 kinase beta-1 and GAPDH—Glyceraldehyde-3-phosphate dehydrogenase targets. The inventors first scraped the RSCB PDB for the PDB IDs of the structures belonging to all the different UNIPROT IDs in the assay. The number of PDB IDs found for a particular UNIPROT varied (P00533 from the EGFR assay has 260). Complete sets were analyzed and the average as well as minimum values of the docking scores calculated for each of the assays over all the entries in the complete docked database. The resulting heatmap plot of the average values of the docking scores is shown in FIG. 5. The inventors demonstrate that the interaction patterns are distinct for the individual cannabinoids on the examined assay set. Similar observations can be observed in in-vitro cell-based and reporter assays as well. For example, CBLA gives the lowest docking scores (indicating a favorable binding potential) with the proteins from the assay. Similarly behaved is THCA (also indicating a favorable binding potential). On the other hand CBCA and THCV both have the least favorable interaction on the assay target set (indicating a weaker binding potential than CBLA or THCA with the proteins of the assay).

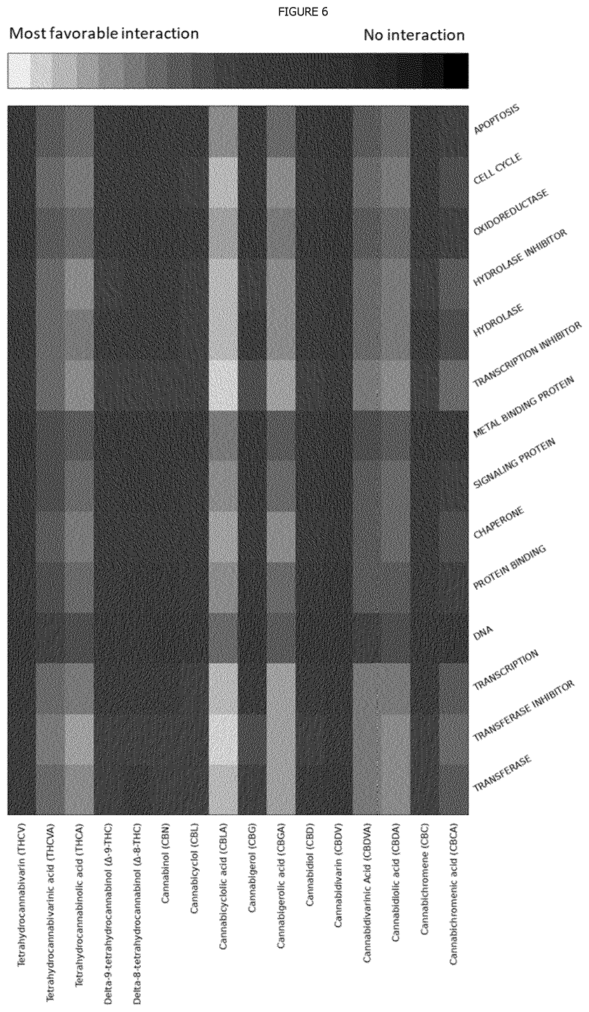

Furthermore, the assay systems were classified. FIG. 6 shows a heatmap of the average docking scores for classifications across the whole docked database, with a focus on the classifications of the assay proteins. FIG. 7 shows a heatmap of the average docking scores for classifications across the whole docked database, with a focus on the classifications of the assay receptor proteins The inventors thus demonstrate distinct binding patterns for individual cannabinoids and a support the rationale of forming cannabinoid formulations that can support a complex biological response supported by cell (viability) studies, biomarker analysis, reporter assays, transcriptome and expression studies.

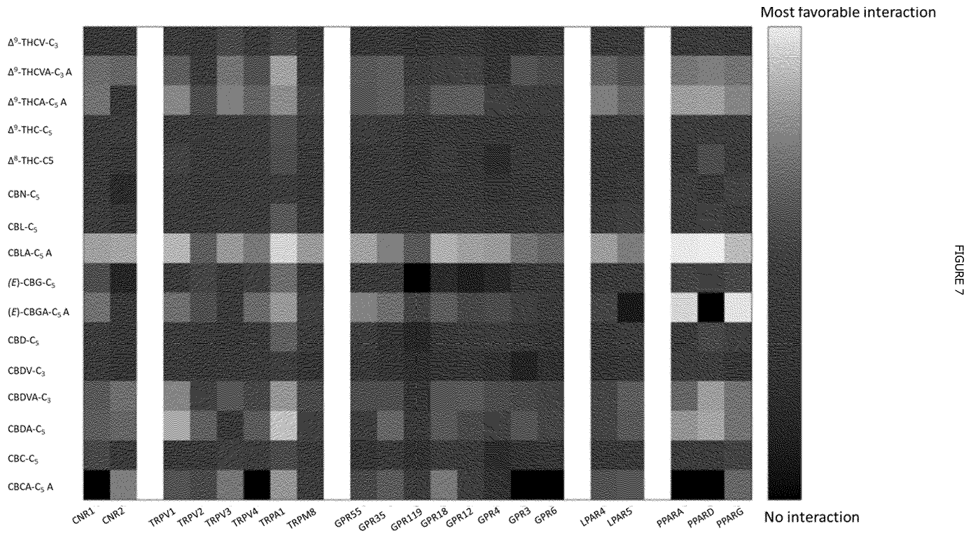

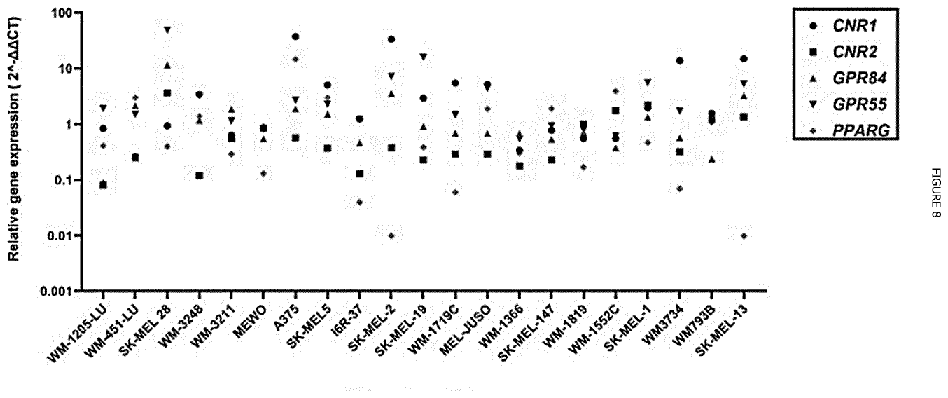

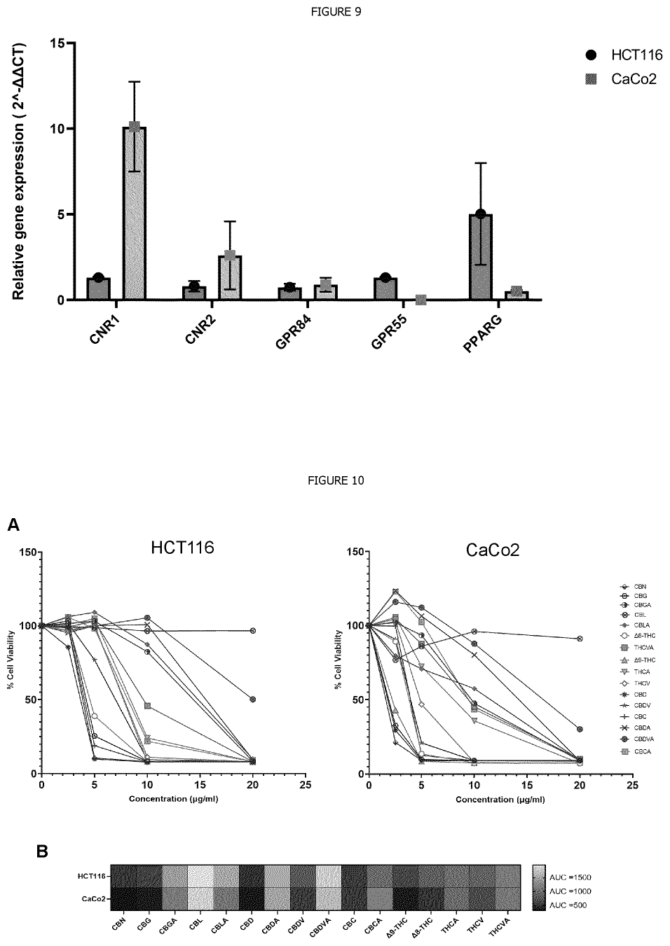

Example 4: In a preliminary analysis, the inventors investigated the gene expression of 5 known cannabinoid receptors (CNR1, CNR2, GPR84, GPR55, PPARγ) in the panel of melanoma cells (FIG. 8) and the colon cancer cell lines CaCo2 and HCT116 (FIG. 9). In case of the panel of melanoma cell liens, the inventors observed that expression of selected cannabinoid targets is heterogeneously expressed across the cell panel and within the specific cell lines, pointing towards the variable expression of cannabinoid targets. In the case of the colon cancer cell lines CaCo2 and HCT116, Cannabinoid receptor 1 (CN1R) mRNA expression was significantly higher in the CaCo2 cell line compared with HCT116. Cannabinoid receptor 2 (CNR2) expression was also higher in CaCo2 but did not reach statistical significance. Expression of G protein-coupled receptor 55 (GPR55), sometimes referred to as CB3, was detected in HCT116 but not in CaCo2. Expression of G protein-coupled receptor 84 (GPR84) was detected in both cell lines, but at low levels, whereas expression of peroxisome proliferator-activated receptor gamma (PPARγ) was significantly higher in the HCT116 cell line. These findings suggest that the expression of cannabinoid receptors vary between different types of cancer cells, and that this could potentially result in variable response to cannabinoid-based therapies.

Example 5: In a preliminary analysis, the inventors investigated the effect of 16 cannabinoid compounds (Δ9-THCV-C3, Δ9-THCVA-C3 A, Δ9-THCA-C5 A, Δ9-THC-C5, Δ8-THC-C5, CBN-C5, CBL-C5, CBLA-C5 A, (E)-CBG-C5, (E)-CBGA-C5 A, CBD-C5, CBDV-C3, CBDVA-C3, CBDA-C5, CBC-C5, and CBCA-C5 A) on cell viability/proliferation in two colorectal carcinoma cell lines (HCT116 and CaCo2) (FIG. 10). It was observed that the response of cell lines to cannabinoids is heterogeneous with CBN, CBG, CBD, CBC, and Δ8THC and Δ9THC showing the strongest effect in reducing cell viability. CaCo2 cell line showed stronger response compared to HCT116 across most of the tested cannabinoids. This suggests that the cannabinoid compounds have different effects on cell viability/proliferation in these two cell lines, and that the sensitivity to the compounds vary between different types of cancer cells.

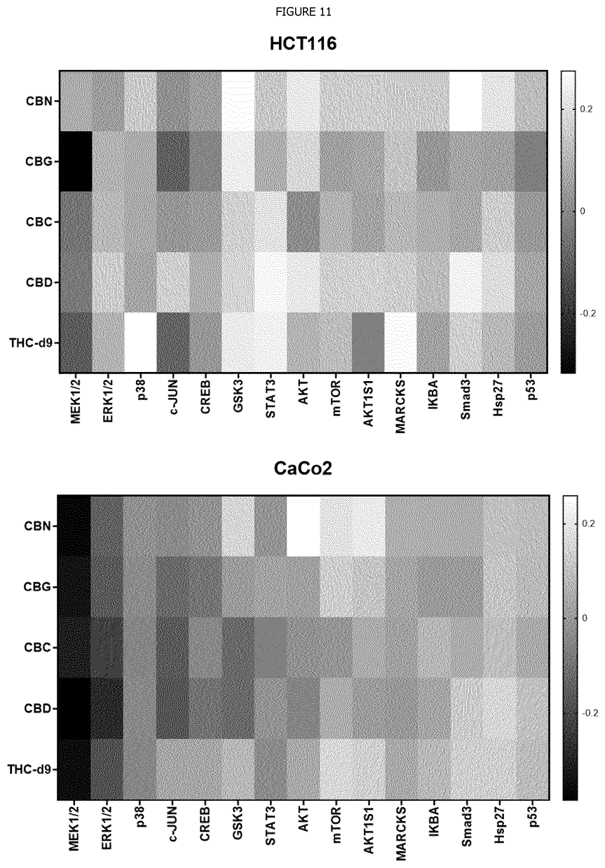

Example 6: In a preliminary analysis, the inventors investigated the expression of 15 phosphoproteins involved in endocannabinoid signaling network (MEK1/2, ERK1/2, P38, JUN, CREB, GSK3, STAT3, AKT, mTOR, AKT1S1, MARCKS, IKBA, SMAD3, HSP27, P53) in response to 5 cannabinoids (CBN, CBG, CBD, CBC, THC). It was observed that expression of phosphoproteins that stimulate cell growth was decreased in response to cannabinoids in CaCo2 cell line to a greater extent than in HCT116 cell line (FIG. 11). Furthermore, the expression of some pro-apoptotic and tumor suppressor proteins was shown to increase in response to cannabinoids in CaCo2 cell line. This finding suggests that the cannabinoids have different effects on endocannabinoid signaling and cell growth in these two cell lines that may be due to variations in the endocannabinoid system between the two cell lines or differences in the specific receptors that are activated by the different cannabinoids.

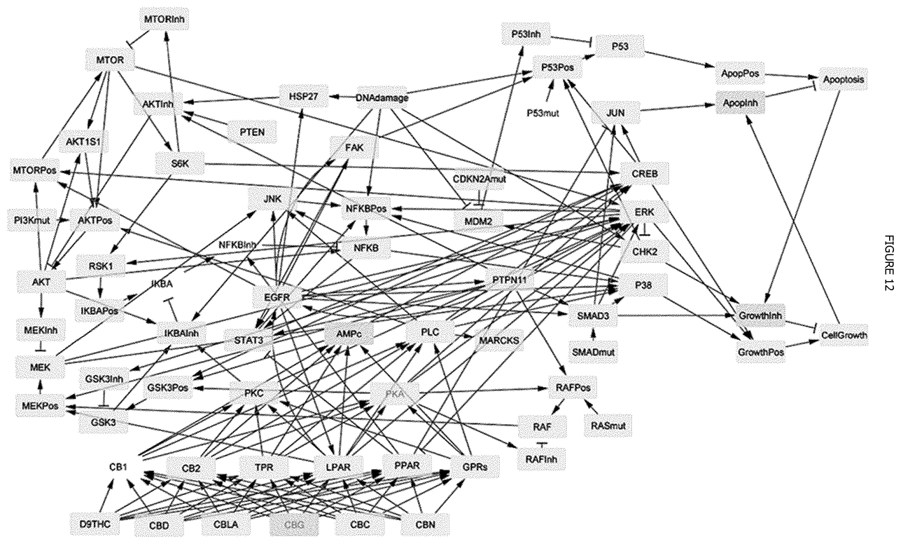

Example 7: In silico model of the ECS signaling network in cancer (FIG. 12). The mathematical model is based on existing literature knowledge of the ECS signaling network and the molecular mechanisms underlying cancer progression. The model uses Boolean logic to represent the interactions between different components of the ECS signaling network, such as receptors, enzymes, and signaling molecules. The model also includes information about the effects of cannabinoids and other modulators on the ECS signaling network. The model was developed to simulate the behavior of the ECS signaling network in different types of cancer cells and tissues under different conditions, such as cannabinoid treatment or genetic mutations. The model can be used to predict the effects of different cannabinoids on the behavior of the ECS signaling network, cancer progression, and identification of potential new targets.

### REFERENCES

The references are:
(1) Nomura D K, et al. Cell. 2010; 140(1):49-61.
(2) Velasco G, et al. Handb Exp Pharmacol. 2015; 231:449-72.
(3) Zheng D, et al. Cancer Res. 2008; 68(10):3992-8.
(4) Joosten M, et al. Exp Hematol. 2002; 30(2):142-9.
(5) Blazquez C, et al. Cancer Res. 2004; 64(16):5617-23.
(6) Blazquez C, et al. Cancer Res. 2008; 68(6):1945-52.
(7) Qamri Z, et al. Mol Cancer Ther. 2009; 8(11):3117-29.
(8) Ramer R, et al. J Natl Cancer Inst. 2008; 100(1):59-69.
(9) Rozanc et al, Plants. 2021; 10(3), 566
(10) Patel B, et al. Journal of Pharmaceutical and Biomedical Analysis. 2017; 146:15-23.
(11) Bolick N L, et al. Hematology/Oncology Clinics. 2021; 35(1):57-72.
(12) Armstrong J L, et al. Journal of Investigative Dermatology. 2015; 135(6):1629-37.
(13) Blazquez C, et al. Faseb J. 2006; 20(14):2633-5.
(14) Zhou J, et al. Oncotarget. 2017; 8(34):56868-79.
(15) Zaiachuck et al. Frot Med (Lausanne). 2021; 8:713153
(16) Konc, et al. J Chem Inf Model. 2022, 62, 6, 1573-1584 (doi: 10.1021/acs.jcim.1c01176)

1. A method for identifying a therapeutic composition against cancer comprising at least one cannabinoid, wherein the method comprises the steps:
(i) determining a therapeutic efficacy of the composition comprising at least one cannabinoid with a predetermined chemical profile,
(ii) performing a computational analysis on the therapeutic efficacy of the composition comprising at least one cannabinoid with the predetermined chemical profile.

2. The method of claim 1, wherein the determining a therapeutic efficacy comprises comparing a biological marker of a sample that is not exposed to the composition comprising at least one cannabinoid to the biological marker of a sample that is exposed to the composition comprising at least one cannabinoid, wherein the increase and/or decrease of the biological marker is indicative of a therapeutic effect.

3. The method of claim 1, wherein the comparing a biological marker comprises conducting a multiomic analysis, preferably wherein the multiomic analysis comprises a cell profiling of the sample, a cell composition analysis, a gene expression analysis, a neighborhood analysis, a cytokine expression profiling and/or a protein phosphorylation analysis.

4. The method of claim 1, wherein the computational analysis comprises correlating the predetermined chemical profile with the therapeutic efficacy of the composition comprising at least one cannabinoid to create a central database and/or wherein the computational analysis is based on machine learning and/or statistical analysis.

5. The method of claim 1, wherein the computational analysis comprises the use of an in silico model, wherein the in silico model describes docking scores or energy calculations of cannabinoids on a potential therapeutic cannabinoid target or target ensemble, metabolic pathways and/or gene expression of a subject or of a cell, when exposing the subject or the cell to the composition comprising at least one cannabinoid.

6. The method of claim 1, wherein the multiomic analysis comprises cell profiling of the sample using a multiplexed immunofluorescence method wherein the multiplexed immunofluorescence method comprises a detection or a quantification, of a cannabinoid-related receptor selected from a receptor class of the group consisting of hydrolase, transferase, oxidoreductase, signaling protein, cell adhesion protein, transport protein, nuclear receptor, metalloprotease, ligase, matrix metalloprotease, flavoprotein, cell cycle related protein, lyase, isomerase, protein involved in immunological response, hormone receptor, protein involved in blood, transcription and apoptotic protein.

7. The method of claim 6, wherein the cannabinoid-related receptor is selected from at least one cannabinoid-related receptor of the group consisting of CBR1 (also called CNR1, CB1), CBR2 (also called CB2, CNR2) TRPV1, TRPV2, TRPV3, TRPV4, TRPA1, TRPVA1, TRPM8, GPR3, GPR6, GPR12, GPR18, GPR23 (also called LPAR4), GPR35, GPR55, GPR84, GPR92 (also called LPAR5), GPR119, PPARα (also called PPARA), PPARβ (also called PPARD) and PPARγ (also called PPARG).

8-9. (canceled)

10. The method of claim 3, wherein the cytokine expression profiling comprises a detection or a quantification, of at least one cytokine selected from the structural families of four-α-helix bundle, IL-1, cysteine knot cytokines and IL-17 family and/or at least one secretory cytokine selected from the group consisting of EGF, FGF, VEGF, TNF, IFN, TGF, TRAIL, SCF, MSP, CD, OSM, BBL, GITRI, LIGHT, OX, TALL, TWEAK, TRANCE and Interleukins.

11. (canceled)

12. The method of claim 3, wherein the protein phosphorylation analysis comprises conducting a multiplex ELISA, and/or
wherein the protein phosphorylation analysis comprises a detection or a quantification, of the phosphorylation of at least one intracellular signaling protein selected from the group consisting of MEK1, ERK1/2, AKT, FAK1, CHK2, JUN, CREB1, EGFR, RB, GSK3, HSP27, P38, IκB, NF-κB, mTOR, RSK1, SMAD3, AKT1S1, CHK2, MARCKS, p70S6K, LCK, PI3K, P53, PTN11, NRF2, STAT1, STAT3, STAT6, SRC and/or WNT, or
wherein the protein phosphorylation analysis comprises a detection, optionally a quantification, of the phosphorylation of at least one intracellular signaling protein selected from the group consisting of MEK1/2, ERK1/2, P38, JUN, CREB, GSK3, STAT3, AKT, mTOR, AKT1S1, MARCKS, IKBA, SMAD3, HSP27 and P53.

13-15. (canceled)

16. The method of claim 1, wherein the computational analysis comprises the creation of a database of potential therapeutic cannabinoid targets.

17. (canceled)

18. The method of claim 10, wherein the database of potential therapeutic cannabinoid targets comprises AKT, CREB1, GSK3, Hsp27, IKBA, MEK1/2, mTOR, p38, MAPK, p53, RSK1, Smad3, AKT1S1, CHK2, cJUN, EGFR, p44/42 MAPK (ERK1/2), MARCKS, NF-κB, STAT3, PTN11, FAK, JNK, p70S6K and/or GAPDH.

19. The method of claim 10, wherein the database of potential therapeutic cannabinoid targets is created by the use of in silico target prediction methods selected from SwissTarget, BioGRID, canCAR and/or the CaNDis web server and/or wherein the computational analysis comprises steps for identifying potential therapeutic cannabinoid targets comprising:
matching of a potential therapeutic cannabinoid target with its gene data,
matching a potential therapeutic cannabinoid target by its presence in key biological pathways, and/or
matching of a potential therapeutic cannabinoid target with its gene and expression data by using deep-learning type models, using Pythorch, KERAS and/or TensorFlow libraries.

20-22. (canceled)

23. The method of claim 5, further comprising studying the interaction of cannabinoids, and their metabolites with the potential therapeutic cannabinoid targets, wherein the interaction of the cannabinoids and/or their metabolites with their potential therapeutic cannabinoid targets is evaluated by using direct methods, inverse molecular docking, and/or by constructing drug-target interaction fingerprints, maps and/or structure-activity relationships.

24-27. (canceled)

28. The method according to claim 1, comprising setting up mechanistic models of action of individual cannabinoids and/or combinations of cannabinoids.

29. A therapeutic composition against cancer comprising at least one cannabinoid.

30. (canceled)

31. The therapeutic composition against cancer comprising at least one cannabinoid of claim 29, wherein the cannabinoid is a phytocannabinoid (pCB) and/or the at least one cannabinoid is selected from the group consisting of Δ9-THCV-C3, Δ9-THCVA-C3 A, Δ9-THCA-C5 A, Δ9-THC-C5, Δ8-THC-C5, CBN-C5, CBL-C5, CBLA-C5 A, (E)-CBG-C5, (E)-CBGA-C5 A, CBD-C5, CBDV-C3, CBDVA-C3, CBDA-C5, CBC-C5, and CBCA-C5 A.

32-34. (canceled)

35. A method for the treatment of a condition or disease, wherein the treatment comprises administration of the therapeutic composition against cancer comprising at least one cannabinoid of claim 29, wherein the treatment targets the endocannabinoid system.

36. The method of claim 35, wherein the disease is cancer.

37-38. (canceled)

39. An in vitro method for the personalization of cannabis-based cancer therapy, wherein the method comprises the steps:
(a) providing a patient sample,
(b) performing a multiomic analysis on said patient sample, wherein the multiomic analysis comprises detection, optionally a quantification, of a biological marker according to claim 2
(c) predicting the patient's response to a therapeutic composition comprising at least one cannabinoid, wherein said step comprises comparing the data generated by step (b) to library data, wherein the library data is a central database correlating a predetermined chemical profile with a therapeutic efficacy of the therapeutic composition comprising at least one cannabinoid or a database of potential therapeutic cannabinoid targets created by a computational analysis on a therapeutic efficacy of the composition comprising at least one cannabinoid with a predetermined chemical profile, and
(d) selecting a therapeutic composition comprising at least one cannabinoid for treatment of the patient based on the prediction of the patient's response.

40-45. (canceled)

46. The therapeutic composition against cancer comprising at least one cannabinoid of claim 29, wherein the therapeutic composition against cancer comprising at least one cannabinoid is obtainable by:
determining a therapeutic efficacy of the composition comprising at least one cannabinoid with a predetermined chemical profile,
performing a computational analysis on the therapeutic efficacy of the composition comprising at least one cannabinoid with the predetermined chemical profile.
